(19) United States
(12) Patent Application Publication
(10) Pub. No.: US 2021/0317410 A1
(43) Pub. Date: Oct. 14, 2021

(54) METHODS OF MAKING NATURAL KILLER CELLS AND USES THEREOF

(71) Applicant: CoImmune, Inc., Durham, NC (US)

(72) Inventors: Mark DEBENEDETTE, Durham, NC (US); Joseph HORVATINOVICH, Durham, NC (US); Charles NICOLETTE, Durham, NC (US)

(21) Appl. No.: 17/058,430

(22) PCT Filed: May 24, 2019
(86) PCT No.: PCT/US2019/033950

§ 371 Date: Nov. 24, 2020

Related U.S. Application Data
(60) Provisional application No. 62/677,019, filed on May 27, 2018.

(51) Int. Cl. C12N 5/0783; A61K 35/15; C07K 16/28; A61K 39/395; G01N 33/569

(52) U.S. Cl. CPC C12N 5/0646; A61K 35/15; C07K 16/283; G01N 2800/52; G01N 33/56972; C12N 2501/998; C12N 2502/1121; A61K 39/3955

(57) ABSTRACT

The present invention relates to Natural Killer (“NK”) cells and uses thereof. In some embodiments NK cells are generated in vitro and used for adoptive transfer therapy. In some embodiments NK cells are generated in vivo to stimulate a patient's immune response.

### FIELD OF THE INVENTION

The present invention relates to Natural Killer (“NK”) cells and uses thereof. In some embodiments NK cells are generated in vitro and used for adoptive transfer therapy. In some embodiments NK cells are generated in vivo to stimulate a patient's immune response.

### BACKGROUND

Cell therapy utilizes modified antigen presenting cells (APCs) or immune effector cells to initiate an immune response in a patient. Antigen presenting cells are central to cell therapy because they initiate the immune response; specifically, they are capable of inducing a primary immune response from T lymphocytes. Dendritic cells (DCs) are the most potent APCs involved in adaptive immunity; they coordinate the initiation of immune responses by naive T cells and B cells and induce antigen-specific cytotoxic T lymphocyte (CTL) responses.

DCs are specialized in several ways to prime helper and killer T cells in vivo. For example, immature DCs that reside in peripheral tissues are equipped to capture antigens and to produce immunogenic MHC-peptide complexes. In response to maturation-inducing stimuli such as inflammatory cytokines, immature DCs develop into potent T-cell stimulators by upregulating adhesion and costimulatory molecules and migrate into secondary lymphoid organs to select and stimulate rare antigen-specific T cells. However, potent stimulation of T cells occurs only after DC maturation, a process that increases the availability of MHC-peptide complexes on the cell surface in addition to co-stimulatory molecules that direct the effector function of the responding T-cells.

Co-stimulation is typically necessary for a T cell to produce cytokine levels sufficient to induce clonal expansion. Dendritic cells are rich in co-stimulatory molecules of the immune response, such as the molecules CD80 and CD86, which activate the molecule CD28 on T lymphocytes. In return, T-helper cells express CD40L, which ligates CD40 on DCs. These and other mutual interactions between DCs and T cells lead to ‘maturation’ of the DCs and the development of effector function in the T cells. The expression of adhesion molecules such as, for example, the molecule CD54 or the molecule CD11a/CD18 facilitate cooperation between the DCs and the T cells.

Another special characteristic of DCs is to deploy different functions depending on their stage of differentiation. Thus, the capture of antigen and its transformation are the two principal functions of the immature dendritic cell, whereas the capacity of a DC to present antigen for T-cell stimulation increases as the DCs migrate into the tissues and the lymphatic ganglia. This change of functionality corresponds to a maturation of the dendritic cell. In this manner, the development of an immature dendritic cell into a mature dendritic cell represents a fundamental step in the initiation of the immune response.

Traditionally, this maturation was followed by monitoring changes of the surface markers on the DCs during the process. Some cell surface markers characteristic of different stages of DC maturation include: CD34+ for hematopoietic stem cell; CD14++, DR+, CD86+, CD16+/−, CD54+, and CD40+ for monocytes; CD14+/−, CD16−, CD80+/−, CD83−, CD86+, CD1a+, CD54+, DQ+, DR++ for immature dendritic cells, and CD14−, CD83++, CD86++, CD80++, DR+++, DQ++, CD40++, CD54++, CD1a+/− for mature dendritic cells, where “+” indicates positive expression, “++” indicates higher expression, “+/−” indicates weaker expression, and “−” indicates very weak or undetectable expression. However, the surface markers can vary depending upon the maturation process.

It is difficult to isolate mature dendritic cells from peripheral blood because they comprise less than 1% of the white blood cells, and mature DCs are also difficult to extract from tissues. This difficulty, in combination with the potential therapeutic benefit of DCs in cell therapy, has driven research and development toward new methods to generate mature dendritic cells using alternative sources. Several methods are reported to produce mature DCs from immature dendritic cells, and it has been shown that different methods can produce mature DCs with different properties. Methods that produce mature DCs with particularly advantageous properties include those disclosed in WO2006042177 (Healey et al.); WO2007117682 (Tcherepanova et al.); DeBenedette et al. (2008) J. Immunol. 181: 5296-5305; and Calderhead et al. (2008) J. Immunother. 31: 731-41, herein incorporated by reference in their entireties. These methods in particular produce mature DCs designated “PME-CD40L” DCs. It has been shown that PME-CD40L DCs can be used to treat a human patient having an immune disease or disorder and also to stimulate the production in vivo of advantageous T cells.

PME-CD40L DCs can be produced, for example, by a method comprising the sequential steps of: (a) culturing isolated immature dendritic cells (iDCs) with an interferon gamma receptor (IFN-γR) agonist in the presence of a TNF-αR agonist and PGE₂ for approximately 12 to 30 hours to produce CD83⁺ mature dendritic cells; and (b) transfecting said CD83⁺ mature dendritic cells (mDCs) with a CD40 agonist to produce a transient CD40 signal. In some embodiments, the CD40 agonist is mRNA (messenger RNA) encoding a CD40L polypeptide. In some of these embodiments, the mRNA encodes a CD40L polypeptide consisting of amino acid residues 21-261 of SEQ ID NO:2 of WO2007117682.

In some embodiments, the mRNA encoding the CD40L polypeptide may be cotransfected with an mRNA encoding an antigen. PME-CD40L DCs are mature DCs that are also phenotypically CD83⁺ and CCR7⁺. PME-CD40L DCs stimulate various immune responses in patients treated with them, including the production of “stem cell memory” T cells, also designated “T_{SCM}” cells, as described, for example, in WO 2015/127190.

Argos Therapeutics has been conducting a phase III clinical trial of treatment with AGS-003 (the “ADAPT” trial), a therapy comprising PME-CD40L DCs. Results from an earlier phase II clinical trial were described, for example, in Amin et al. (2015) J. Immunother. Cancer 3: 14.

### SUMMARY OF THE INVENTION

The instant inventors have surprisingly discovered that different patients can have qualitatively and quantitatively different responses to treatment with PME-CD40L DCs. In the phase III clinical trial of treatment with AGS-003 (the “ADAPT” trial), a therapy comprising PME-CD40L DCs, some patients exhibited significant improvement in certain indicators of immune response while others either did not exhibit any improvement or exhibited improvement in a smaller subset of indicators. The inventors have discovered that some patients who have positive clinical responses after treatment with PME-CD40L DCs exhibit improvement in one or more treatment indicators, such as, for example, an increase in activated Natural Killer (NK) cells and/or an increase in activated NK cells that is correlated with an increase in PD-1+ CD4 T cells.

Patients who had positive clinical responses following treatment with PME-CD40L DCs often also exhibited a correlation between the number of NK cells and PD-1+ CD4 T cells prior to treatment with PME-CD40L DCs. In this manner, the invention provides methods of treating a patient with dendritic cell therapies comprising determining whether the patient's ratio of NK cells to PD-1+ CD4 T cells meets or exceeds a threshold value.

The instant inventors also surprisingly discovered that dendritic cells (DCs) can stimulate the binding of immune complexes (ICs) to CD4 T cells, but this binding can be prevented by an antibody that blocks the binding of ICs to the cell surface receptor CD16. Further, the presence of such antibodies (i.e., antibodies blocking IC binding to CD16) contemporaneously with DCs in cultures of PBMCs was discovered to stimulate the immune response, for example, by increasing the production of activated NK cells and activated functional PD-1+ CD4 T cells. This immune stimulation provided by the contemporaneous presence of DCs and antibodies blocking the binding of ICs to CD16 exhibits synergy, i.e., the effect produced by the contemporaneous presence of DCs and said antibodies produces a combined effect that is greater than the sum of their separate effects.

In this manner, the invention provides methods of stimulating an immune response in a patient or treating a patient comprising administering to said patient DCs and further administering to said patient an antibody or other agent that blocks the binding of ICs to CD16 so that the DCs are present in tissues simultaneously with said antibody or other agent (also referred to herein as “contemporaneous administration”). To achieve this end, the administration of anti-CD16 antibody or other agent to the patient can be simultaneous with administration to the patient of the DCs, or can occur prior to or after administration of the DCs, so long as the DCs and anti-CD16 antibody or other agent are present in the tissues simultaneously. In some embodiments, the DCs and anti-CD16 antibody or other agent are present in the tissues for at least a day, or at least an hour, or at least an amount of time sufficient for at least one indicator of immune response in the patient to be affected.

The invention also provides methods of producing activated NK cells in vitro comprising incubating PBMCs in the presence of both DCs and anti-CD16 antibodies or other agents. These methods can be used to produce populations of activated NK cells in vitro for use in cell transfer therapy to patients, including autologous or heterologous cell transfer therapy, or for other uses. In some embodiments, the activated NK cells are further purified from the cell culture in which they were produced or are further enriched to provide a cell population enriched in NK cells prior to use in adoptive transfer therapy.

### BRIEF DESCRIPTION OF THE FIGURES

FIGS. 1A, 1B, 1C, 1D, 1E, 1F, and 1G show the identification of activated NK cells and functional helper CD4 T cells in mRCC patient PBMCs stimulated with autologous DC product using multi-color flow cytometry (see Example 2).

FIG. 2 shows data demonstrating that mRCC Clinical Responder (“CR”) patients treated with AGS-003 exhibited higher numbers of both CD4 helper T cells and activated NK cells following treatment (“Post”) versus prior to treatment (“Pre”), whereas Non-Responder patients (“NR”) did not show an increase in activated NK cells (see Example 2).

FIGS. 3A, 3B, 3C, and 3D present data showing a positive correlation between the number of activated NK cells and functional CD4 helper T cells in mRCC subjects treated with AGS-003 who had a measurable clinical response (“CR”) whereas patients who did not have a clinical response (“NR”) did not exhibit this correlation. (see Example 2).

FIGS. 4A, 4B, 4C, 4D, 4E, 4F, 4G, 4H, 4I, 4J, 4K, and 4L present data showing that blocking Ig complex binding to CD4 T cells with anti-CD16 antibody during in vitro DC stimulation induces activated NK cells (see Example 3).

FIGS. 5A, 5B, 5C, and 5D present data showing that monocyte killing in activated cultures is specific to CD16 activation and is cell driven (see Example 3).

FIG. 6 presents data showing that a combination of anti-CD16 antibody and DCs increases IFN-γ (“IFNg”) secretion in PBMC cultures. The cultures were unstimulated (“None/none”), stimulated with 3G8 only (“None/3G8”), DCs only (“DC/none”) or a combination of 3G8 and DCs (“DC/3G8”) by cytokine bead array (see Example 3).

FIGS. 7A, 7B, and 7C present data showing that dendritic cell (DC) stimulation of PBMC cultures resulted in the binding of immune complexes (ICs) to the cell surface of CD4 T cells (see Example 3). FIG. 7C shows measuring this binding of ICs to CD4 T cells by mean fluorescence intensity (MFI). Actual MFI values: anti-CD16 antibody blocking ICs binding, MFI value of 315; background values detected on unstimulated PBMCs, MFI value of 418; PBMCs stimulated with DCs (“DC only”), MFI value of 899.

FIG. 8 presents data showing that, even after the addition of the 3G8 anti-CD16 antibody that blocks binding of IgG to the CD16+ cells, CD16+ cells can still be detected with a different antibody (here, the eBioCB16 antibody).

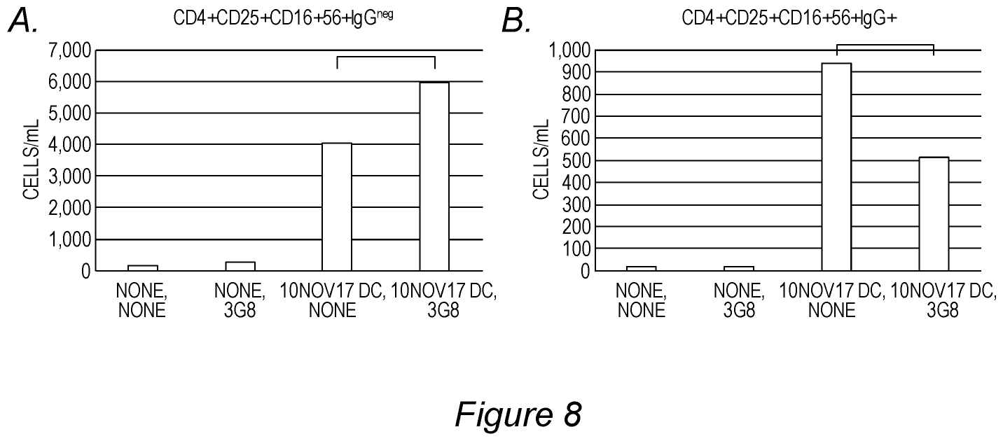

### DETAILED DESCRIPTION OF THE INVENTION

Previously, novel methods were developed to produce mature DCs that are described in detail in: WO2006042177 (Healey et al.); WO2007117682 (Tcherepanova et al.); DeBenedette et al. (2008) J. Immunol. 181: 5296-5305; and Calderhead et al. (2008) J. Immunother. 31: 731-41. In some of these methods, immature DCs are sequentially signaled with a first signal (an IFN-γ receptor agonist and/or a TNF-α receptor agonist) to produce CD83⁺ CCR7⁻ mature DCs and then are signaled with a second signal (a CD40 agonist), producing CD83⁺ CCR7⁺ mature DCs; various IFN-γ receptor agonists and/or TNF-α receptor agonists may be used.

In a method called the “PME-CD40L process” (for Post Maturation Electroporation with CD40L), immature DCs are first cultured in the presence of IFN-γ and TNF-α; optionally, PGE₂ is also added. Then, approximately 12-30 hours later (in some embodiments about 18 hrs later), the cells are electroporated with CD40L mRNA and, optionally, antigen-encoding mRNA. This “PME-CD40L process” produces CD83⁺ CCR7⁺ mature DCs. Cells harvested from this process after electroporation (e.g., 4 hrs post electroporation) and formulated as a vaccine were shown to mediate maximum immunopotency in in vitro assays.

Dendritic cells made by the PME-CD40L process (herein, “PME-CD40L DCs”) are phenotypically different than previously known dendritic cells. For example, PME-CD40L DCs can support long term antigen-specific CTL effector function and induce a type of effector memory CTLs that retain the capacity to expand, produce cytokines, and kill target cells—all critical events in mediating robust long-term CTL effector function.

In some embodiments, methods for producing PME-CD40L DCs comprise the sequential steps of: (a) signaling isolated immature dendritic cells (iDCs) with a first signal comprising an interferon gamma receptor (IFN-γR) agonist, and optionally a TNF-αR agonist, to produce IFN-γR agonist signaled dendritic cells; and (b) signaling said IFN-γR agonist signaled dendritic cells with a second transient signal comprising an effective amount of a CD40 agonist to produce CD83⁺ CCR7⁺ mature dendritic cells. In some embodiments, the CD83⁺ CCR7⁺ mature DCs transiently express CD40L polypeptide; in some instances, the CD40L is predominantly localized intracellularly rather than on the cell surface. At least 60%, at least 70%, at least 80% or at least 90% of the CD40L polypeptide may be localized intracellularly.

PME-CD40L DCs exhibit some distinctive characteristics, including: (i) they demonstrate elevated cell surface expression of co-stimulator molecules CD80, CD83, and CD86; ii) they are CCR7+; and iii) they secrete IL-12 p70 polypeptide or protein, and/or secrete significantly reduced levels (0 to 500 μg per ml per million DCs) of IL-10 (see, e.g., data and experiments presented in WO2006042177 (Healey et al.) and WO2007117682 (Tcherepanova et al.)). In some embodiments, these mature CD83+ CCR7+ DCs produce at least 1000 pg IL-12 per 106 DCs. IL-10 and IL-12 levels can be determined, for example, by ELISA of culture supernatants collected at up to 36 hrs post induction of DC maturation from immature DCs (Wierda et al. (2000) Blood 96: 2917; Ajdary et al. (2000) Infection and Immunity 68: 1760). One of skill in the art can determine when PME-CD40Ls have been produced by sampling a cell or small (sub)population of DCs from a cell population for the presence of mature DCs expressing CD40L mRNA and/or CD40L polypeptide, or expressing interleukin 12 (IL-12) p35 protein. Other characteristics of these cells are discussed, for example, in WO2006042177 (Healey et al.); WO2007117682 (Tcherepanova et al.); DeBenedette et al. (2008) J. Immunol. 181: 5296-5305; and Calderhead et al. (2008) J. Immunother. 31: 731-41.

Immature DCs used to produce PME-CD40L DCs can be isolated or prepared from a suitable tissue source containing DC precursor cells and differentiated in vitro to produce immature DCs. For example, a suitable tissue source can be one or more of: bone marrow cells; peripheral blood progenitor cells (PBPCs); peripheral blood stem cells (PBSCs): and cord blood cells. Preferably, the tissue source is a peripheral blood mononuclear cell (PBMC). The tissue source can be fresh or frozen and can be pre-treated with an effective amount of a growth factor that promotes growth and differentiation of non-stem or progenitor cells, which are then more easily separated from the cells of interest. These methods are known in the art and described briefly, for example, in Romani et al. (1994) J. Exp. Med. 180: 83 and Caux et al. (1996) J. Exp. Med. 184: 695. The immature DCs can also be isolated from peripheral blood mononuclear cells (PBMCs) which optionally are treated with an effective amount of granulocyte macrophage colony stimulating factor (GM-CSF) in the presence or absence of interleukin 4 (IL-4) and/or IL-13, so that the PBMCs differentiate into immature DCs. In some embodiments, PBMCs are cultured in the presence of GM-CSF and IL-4 for about 4-7 days, preferably about 5-6 days, to produce immature DCs. In some embodiments, the first signal is given at day 4, 5, 6, or 7, or at day 5 or 6. In addition, GM-CSF as well as IL-4 and/or IL-13 may be present in the medium at the time of the first and/or second signaling.

To increase the number of dendritic precursor cells in animals, including humans, one can pre-treat subjects with substances which stimulate hematopoiesis. Such substances include but are not limited to G-CSF and GM-CSF. The amount of hematopoietic factor to be administered may be determined by one skilled in the art by monitoring the frequency of cell types in individuals to whom the factor is being administered. U.S. Pat. No. 6,475,483 teaches that dosages of G-CSF of 300 micrograms daily for 5 to 13 days and dosages of GM-CSF of 400 micrograms daily for 4 to 19 days result in significant yields of dendritic cells.

Alternatively, the immature dendritic cells can be signaled with an effective amount of a TNF-α receptor agonist followed by signaling with a CD40 agonist. The immature DCs may be contacted with PGE₂ at about the same time that they receive the first signal of an IFN-γR agonist and a TNF-αR agonist. In some methods, signaling is in the absence of an effective amount of IL-10 and/or IL-6. GM-CSF and at least one of IL-4 or IL-13 may be present in the medium at the time the dendritic cells receive the first and second signals.

Signaling with IFN-γ receptor agonists, TNF-α receptor agonists, and/or CD40 agonists can be accomplished by contacting a cell directly with IFN-γ polypeptides and/or proteins and/or TNF-α polypeptides or proteins and/or CD40 agonists, respectively. Similarly, IFN-γ and TNF-α receptor agonists can be aptamers, antibodies, and the like, that have a similar biological activity. Alternatively, signaling of a cell with IFN-γR agonists, TNF-αR agonists and/or CD40 agonists can occur upon translation of mRNA encoding such polypeptides or proteins within the dendritic cell. Such mRNA may be introduced into the cell by transfection or other means, and the signaling then occurs upon expression of the IFN-γR agonist, TNF-αR agonist and CD40 agonist polypeptides and/or proteins. Thus, signaling can be initiated by providing the signaling agonist in the culture medium, introduction of the agonist into the cell, and/or upon translation within the dendritic cell of an mRNA encoding an agonistic polypeptide. The methods can be practiced in vivo or ex vivo.

Dendritic cells matured ex vivo according to the methods of the invention can then be administered to a subject to induce or enhance an immune response.

Dendritic cells can be further modified by the administration of an immunogen (e.g., an antigen) to the DCs. The immunogen can be delivered in vivo or ex vivo. The immunogen can be delivered to the cells using methods known in the art, and can be delivered as polypeptides or proteins (e.g., by “pulsing”) or as nucleic acids encoding the immunogen (e.g, by transfection or electroporation). In some embodiments, the polynucleotide is an mRNA. In some methods of producing PME-CD40L DCs, the antigen-encoding mRNA is electroporated together with an mRNA encoding a CD40 agonist or substantially concurrent with CD40 agonist signaling.

As will be understood by one of skill in the art, PME-CD40L DCs can also be transfected with RNA encoding antigens from any pathogen of interest; such antigens can be from one individual subject or multiple subjects and can be from a pathogen infection of the subject from which the antigens are isolated or from another subject. Consensus antigens and pathogen-specific antigens are known in the art and may also be used in these methods of preparing PME-CD40L DCs. The DCs will process the antigens and display the antigens on their cell surface; these mature DCs can be used to educate naïve immune effector cells. For example, PME-CD40L DCs loaded with total amplified Renal Cell Carcinoma (“RCC”) tumor RNA induced a fully autologous CTL response (see WO2006042177 (Healey et al.)).

Many methods are known in the art for the isolation and expansion of various cells for in vitro expansion and differentiation into dendritic cells, including CD34⁺ stem cells (see for example, U.S. Pat. No. 5,199,942). The following descriptions are for the purpose of illustration only and in no way are intended to limit the scope of the invention.

CD34⁺ stem cells can be isolated from bone marrow cells or by panning the bone marrow cells or other sources with antibodies which bind unwanted cells, such as CD4⁺ and CD8⁺ (T cells) (see, e.g., Inaba, et al. (1992) J. Ep. Med. 176: 1693-1702). Human CD34⁺ cells can be obtained from a variety of sources, including cord blood, bone marrow explants, and mobilized peripheral blood. Purification of CD34⁺ cells can be accomplished by antibody affinity procedures, for example, as described in Paczesny et al. (2004) J. Exp. Med. 199: 1503-11; Ho et al. (1995) Stem Cells 13 (suppl. 3): 100-105; Brenner (1993) Journal of Hemalotherapy 2:7-17; and Yu. et al. (1995) PNAS 92: 699-703.

CD34⁺ stem cells can be differentiated into dendritic cells by incubating the cells with appropriate cytokines, as is known in the art. For example, human CD34⁺ hematopoietic stem cells can be differentiated in vitro by culturing the cells with human GM-CSF and TNF-α (see, e.g., Szabolcs, et al. (1995) J. Immunol. 154: 5851-5861). Optionally, SCF or other proliferation ligand(s) (e.g., Flt3) is added. Dendritic cells can be isolated from a population of mixed cell types or enriched in a population of cells by florescence activated cell sorting (FACS) or by other standard methods.

As is apparent to those of skill in the art, dose ranges for differentiating stem cells and monocytes into dendritic cells are approximate. Different suppliers and different lots of cytokine from the same supplier vary in the activity of the cytokine. One of skill in the art can readily titrate each cytokine which is used to determine the optimal dose for any particular cytokine.

DCs can be generated from non-proliferating CD14⁺ precursors (monocytes) in peripheral blood by culture in medium containing GM-CSF and IL-4 or GM-CSF and IL-13 (see, e.g., WO 97/29182; Sallusto and Lanzavecchia (1994) J. Exp. Med. 179: 1109 and Romani et al. (1994) J. Exp. Med. 180:83). In some instances, patients can be pretreated with cytokines such as G-CSF, but in most cases this is not necessary because CD14⁺ precursors are sufficiently abundant (Romani et al. (1996) J. Immunol. Methods 196: 137). Others showed that it is possible to avoid non-human proteins such as FCS (fetal calf serum), and to obtain fully and irreversibly mature and stable DCs by using autologous monocyte conditioned medium as maturation stimulus (see, e.g., Romani et al. (1996) Immunol. Methods 196: 137; Bender et al. (1996) J. Immunol. Methods 196: 121). However, these studies did not result in mature DC having increased levels of IL-12 and/or decreased levels of IL-10 and thus did not produce PME-CD40L DCs.

In some embodiments, DCs used for administration to a patient or subject or to produce NK cells in vitro in methods of the invention are derived from the same patient or subject; that is, the DCs and the NK cells or their precursor cells are obtained from the same patient or subject (i.e., they are autologous). In other embodiments, the DCs and the NK cells are derived from different subjects (i.e., they are allogeneic).

In some embodiments, the PME-CD40L DCs used in methods of the invention to produce activated NK cells are transfected with RNA encoding part or all of the HIV proteins Gag, Nef, Tat, and Rev, as described in WO2006031870 and U.S. Pub. No. 20080311155 (Nicolette et al.). Briefly, DCs are transfected with RNA encoding one or more polypeptides from multiple strains of HIV present in an individual subject; the RNA is derived from nucleic acid amplification of pathogen polynucleotides. Primers to amplify such pathogen polynucleotides can be designed to compensate for sequence variability between multiple strains of said pathogen, for example, when said pathogen is HIV, as described in WO2006031870 and U.S. Pub. No. 20080311155. Such primers can include, for example, primers disclosed in WO2006031870, including forward and reverse primers for Gag, Nef, Tat, and Rev. DCs resulting from this process (sometimes referred to as “AGS-004”) have been shown to be capable of stimulating an immune response to HIV in HIV patients (see, for example, Routy and Nicolette (2010) Immunotherapy 2: 467-76).

PME-CD40L DCs can also be stored by contacting an enriched dendritic cell population with a suitable cryopreservative under suitable conditions and frozen, for example, as taught in U.S. Pat. No. 8,574,901.

Experiments conducted by the inventors and described herein in working Examples 2 and 3 show that increases in activated NK cells are linked to better Overall Survival (OS) in mRCC patients receiving DC therapy. Specifically, patients enrolled in the ADAPT study who received 3, 5, or 7 doses of DCs had an increase in activated NK cells which correlated with better overall survival. While the invention is not limited by any particular mechanism of action, it is believed that the mechanism by which DC therapy may activate NK cells is through direct signaling via the CD16 receptor, resulting in increased expression of CD25 (the receptor for IL-2), Grb (involved with lytic activity), and IFN-γ cytokine secretion. Moreover, combination therapy with DCs and anti-CD16 antibody may block binding of natural immune complexes present in the plasma or culture supernatant to CD4 T cells and increase their activity. It may also be the case that blocking IC binding to CD4 T cells disrupts regulatory T cell function and enhances immune activation. Blocking of IC binding in the presence of DC immunotherapy may more efficiently activate CD4 T cell help and subsequently activate NK cells and CD8 T cells.

In this manner, the invention provides methods of stimulating an immune response in a patient or treating a patient comprising administering DCs to said patient and further administering an anti-CD16 antibody or other anti-CD16 agent to said patient so that the DCs are present in tissues simultaneously with the anti-CD16 antibody or agent. To achieve this end, the administration of anti-CD16 antibody or other agent to the patient can be contemporaneous with administration to the patient of the DCs, or can occur prior to or after administration of the DCs, so long as the DCs and anti-CD16 antibody or other agent are present in the tissues simultaneously. In some embodiments, the DCs and anti-CD16 antibody or other agent are present simultaneously in the tissues for at least a day, or at least an hour, or at least an amount of time sufficient for at least one indicator of immune response in the patient to be affected.

The invention also provides methods of producing activated NK cells in vitro comprising incubating PBMCs in the presence of both DCs and anti-CD16 antibodies or other anti-CD16 agents. These methods can be used to produce populations of activated NK cells in vitro for use in cell transfer therapy to patients, including autologous or heterologous cell transfer therapy, or for other uses. In some embodiments, the activated NK cells are further purified from the cell culture in which they were produced or are further enriched prior to use in adoptive transfer therapy.

In some embodiments, PBMCs are isolated from human patients or other mammals and cocultured with PME-CD40L DCs derived from the same patient or subject in the presence of anti-CD16 antibody or other anti-CD16 agent to produce and/or expand NK cells in vitro. These NK cells can then be used for adoptive transfer therapy by infusing them back into the same patient (autologous therapy) or into another patient (allogeneic therapy). Successful allogeneic adoptive transfer therapy of hematopoietic stem cells and lymphocytes has been reported, for example, in Cieri et al. ((2014) Immunol. Rev. 257: 165-180) and Kolb et al. (1995) Blood 86: 2041-50.

In some embodiments, PME-CD40L DCs are loaded with antigen and used to expand a population of NK cells in the presence of anti-CD16 antibody or other agent. The resulting population includes NK cells that are reactive to the antigen; for example, at least 30%, 40%, 50%, 60%, 70%, 80%, or 90% or more of the NK cells produced by the expansion will be reactive to the antigen. In this manner, NK cells can be produced that are reactive to a particular antigen or antigens; these cells can be used to enhance or stimulate the patient's immune response to the antigen by adoptive transfer therapy. For example, antigens can be prepared from a patient's own cancer cells and loaded into DCs that are used to expand a population of NK cells that are then infused back into the patient. In some embodiments, the DCs are provided with antigen(s) by transfecting the DCs with RNA encoding the antigen(s).

NK cells with particular antigen receptors can also be generated using methods of genetic modification, for example, with chimeric antigen receptors as disclosed in Rezvani et al. ((2017) Mol. Ther. 25: 1769-81).

In some embodiments, a patient infected with HIV can be treated by preparing antigens from their HIV-infected cells, loading those antigens into DCs, and using those DCs with an anti-CD16 antibody or other agent to expand a population of NK cells that are infused back into the patient. Antigens can be provided to DCs either by antigen loading or by transfecting the DCs with RNA encoding the antigen(s). Methods for preparing antigens from HIV patients and preparing DCs that present them are known in the art, for example, as described in WO2006031870 (Nicolette et al.) and discussed in more detail elsewhere herein.

In some methods of the invention, HIV patients are treated with AGS-004 and anti-CD16 antibody or agent(s). “AGS-004” refers to PME-CD40L DCs containing “GNVR”, the RNA antigen payload encoding the antigens Gag (G), Nef (N), Vpr (V), and Rev (R) (also called “GNVR DCs”) (as described, for example, in WO2006031870).

In this manner, the invention provides a method of enhancing immunity in a subject comprising administering to the subject an effective amount of NK cells. Introducing or administering immune cells into a subject is generally referred to as adoptive transfer therapy and is intended to help stimulate the subject's immune response. Adoptive transfer therapy is known in the art and has been demonstrated in a number of studies, such as, for example, Cobbold et al. (2005) J. Exp. Med 3: 379-86 and Schmitt et al. (2011) Transfusion 3: 591-99.

The invention provides compositions and methods that can stimulate an immune response in a subject by administering to the subject an effective amount of an enriched population of cells, e.g., NK cells or activated NK cells. The cells can be allogeneic or autologous to the subject. They can be administered to a subject to stimulate or induce an immune response in a subject in a method comprising administering to the subject an effective amount of an enriched population of NK cells.

Thus, the invention also provides compositions and medicaments comprising PBMCs, DCs, and at least one anti-CD16 antibody or agent. These compositions or medicaments can be used to treat a patient in need of stimulation of an immune response, such as, for example, a cancer patient or an HIV patient. In some embodiments, the composition or medicament further comprises a pharmaceutically acceptable carrier.

Compositions or medicaments of the invention and/or cells produced by the methods of the invention can be administered directly to the subject (e.g., a human patient) in any suitable manner. Suitable methods of administering cells to a patient are known in the art. Administration can be by any suitable method that successfully delivers the cell(s) or composition(s) into ultimate contact with a subject's blood or tissue cells. Preferred routes of administration include but are not limited to intradermal and intravenous administration.

Pharmaceutically acceptable carriers are determined in part by the particular medicament or composition being administered, as well as by the particular method used for administration. Accordingly, there is a wide variety of suitable formulations of pharmaceutical compositions and medicaments of the present invention. Most typically, quality controls are conducted and the cells are infused into the patient from whom they were derived and/or isolated, preceded by the administration of diphenhydramine and hydrocortisone; see, for example, Korbling et al. (1986) Blood 67: 529-532 and Haas et al. (1990) Exp. Hematol. 18: 94-98. The dose of cells administered to a subject is in an amount effective to achieve the desired beneficial therapeutic response in the subject over time, or to inhibit growth of cancer cells, or to diminish the activity of a virus infecting the patient (i.e., an “effective amount”).

For the purpose of illustration only, a method of adoptive transfer therapy of the invention can be practiced by obtaining and saving blood samples from the patient prior to infusion for subsequent analysis and comparison. Generally at least about 10⁴ to 10⁶ and typically between 1×10⁸ and 1×10¹¹ cells can be infused (e.g., intravenously or intraperitoneally) into a 70 kg patient over roughly 60-120 minutes. Vital signs and oxygen saturation by pulse oximetry can be closely monitored, and blood samples obtained at intervals following infusion (e.g., 5 minutes and 1 hour) and saved for analysis. Cell re-infusions can be repeated roughly every month for a total of 10-12 treatments in a one-year period, if deemed appropriate. After the first treatment, infusions can be performed on an outpatient basis at the discretion of the clinician. If the re-infusion is given as an outpatient treatment, typically the participant is monitored for at least 4 hours following the treatment.

Compositions, medicaments, and/or cells of the present invention can be administered at a rate determined by the effective dose, the LD-50 (or other measure of toxicity), and the side-effects at various concentrations, as indicated by the mass and overall health of the subject. Administration can be accomplished via single or divided doses. The cells, compositions, and/or medicaments of this invention can supplement other treatments for a condition according to conventional therapy or standard-of-care treatment(s), including cytotoxic agents, nucleotide analogues and biologic response modifiers.

In carrying out the methods of the invention, cell surface markers can be used to identify and/or isolate and/or enrich cells for various purposes. For example, DCs can be distinguished from other cells because they express MHC molecules and costimulatory molecules (e.g., B7-1 and B7-2) and lack markers specific for granulocytes, NK cells, B cells, and T cells. The expression of markers facilitates identification, purification, and separation of these cells from other cells expressing at least one different marker.

Cells can be isolated and/or characterized by flow cytometry methods such as FACS analysis as well as by any suitable method known in the art, including but not limited to methods such as column chromatography. Western blots, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like.

Labeling agents which can be used to label cell antigen include but are not limited to monoclonal antibodies, polyclonal antibodies, proteins, or other polymers such as affinity matrices, carbohydrates or lipids. Detection proceeds by any known method, including for example immunoblotting, Western blot analysis, tracking of radioactive or bioluminescent markers, capillary electrophoresis, or other methods which track a molecule based on size, charge or affinity.

NK cells can be identified by multi-color flow cytometry as cells that are CD3 negative, CCR7 negative, and CD45RA negative. In some embodiments, NK cells can be identified or further identified as cells that are positive for CD16 and/or CD56 expression. NK cells can be identified as activated when they are BrdU⁺ and Granzyme B positive. Further subgating can identify cells that are positive for CD107a, and in this manner, activated NK cells can be identified as BrdU⁺/Grb⁺ (Granzyme B⁺)/CD107a⁺. Thus, the invention also provides methods of identifying NK cells and activated NK cells comprising monitoring and/or identifying cells expressing one or more of CD25, Granzyme b, and CD107a. as well as compositions comprising activated NK cells expressing one or more of CD25, Granzyme b, and CD107a for use in evaluating the immune response of a patient.

For subsequent use in vivo or in vitro, NK cells can also be enriched, isolated or purified from other cells using magnetic bead separation of cells based on the cells expressing or lacking one or more of these markers. Suitable materials and methods are well known in the art, and commercial products and kits are available for many types of cell separation.

Functional helper CD4 T cells can be identified as CD3 positive, CD8 negative, and CD45RA negative. Within the CD8 and CD45RA negative gate, further subgating can identify activated CD4 T cells, defined as PD-1⁺/BrdU⁺. Functional CD4 helper T cells within the PD-1⁺/BrdU⁺ population can also identified by the presence of IL-2. In this manner, functional helper CD4 cells can be identified as CD3⁺/CD8⁻/CD45RA⁻/PD-1⁺/BrdU⁺/IL-2⁺. Depending on the circumstances, one or more of these properties can be used to identify functional CD4 helper T cells.

Regulatory T cells (also referred to herein as “Tregs”) can be identified as CD4⁺ CD25⁺ Foxp3⁺ PD-1⁺ T cells, or as CD4⁺ cells with high levels of expression of CD25 (sometimes called CD25^{hi} or CD25^{high} or CD25⁺⁺). In some embodiments, Tregs can also be identified as one, two, three, four, or five or more of: FoxP3⁺, PD-1⁺, CD45 RA⁺, CD127⁻, CD4⁺, and CD25⁺. In some embodiments, Tregs can alternatively be PD-1⁻ and/or CD45RA⁻.

Cell separation methods based on the expression of surface markers are known in the art and include the use of magnetic bead isolation, FACS sorting (e.g., as discussed in Basu et al. (2010) J. Vis. Exp. 41), and microelectromechanical systems (“MEMS”) chips-based sorting (e.g., as discussed in Shoji and Kawai (2011) Top. Curr. Chem. 2011: 1-25). FACS machines and cell sorters are commercially available (e.g., the BD Bioscience LSRII and the BD FACSAria) and can be used according to manufacturer's instructions.

Cells can be isolated or enriched or separated from other cells by positive or by negative selection where appropriate, or by both positive and negative selection. For example, NK cells can be enriched from a population including other cells such as PBMCs or lymphocytes using positive or negative selection to deplete other cell types. Kits and reagents are known in the art for a variety of purification steps, allowing one of skill in the art to isolate or purify a known cell type; for example, the EasySep™ Human NK cell enrichment kit (STEMCELL Technologies™, Vancouver, BC, Canada). One of skill in the art is capable of selecting particular (often commercially-available) antibodies and selection tools to enrich and/or deplete known cell types from a population of cells.

Selection for cells bearing particular markers can be performed for one marker at a time or for more than one marker at a time (e.g., as discussed in Stemberger et al. (2012) PLoS One 4: e35798). Selection can also be performed serially, and different types of selection can be used on a particular group or population of cells in subsequent selection steps to obtain a desired subpopulation. Cells can also be selected based on their antigen specificity directly by isolating T cells reactive to HLA-peptide complexes (e.g., as discussed in Keenan et al. (2001) Br. J. Haematol. 2: 428-34).

By “anti-CD16 antibody” or “anti-CD16 agent” as used herein is intended an antibody or other agent that interferes with normal CD16 or FcRγIII function. In some embodiments, the anti-CD16 antibody or other agent interferes with normal CD16 function by binding to the Fc portion so as to interfere with immune complex (“IC”) binding. In some embodiments, the anti-CD16 antibody or other agent binds to CD16 but does not bind directly to the Fc portion: in such embodiments, the antibody or other agent prevents IC binding to the CD16 receptor, but binding of monomeric Ig (immunoglobulin) may still occur. In some embodiments, the anti-CD16 antibody is an antibody such as, for example, the 3G8 antibody (Absolute Antibody, Ltd., Oxford, UK) that blocks IC binding but does not interfere with detection of IgG monomers on CD4 T cells. The activity of an anti-CD16 antibody can be measured by alteration of CD16 function in one or more assays known in the art and/or illustrated in the working examples herein.

An anti-CD16 antibody for use in the compositions and methods of the invention can be monoclonal or polyclonal. Anti-CD16 antibodies for use in the compositions and methods of the invention can be antibody derivatives or antibody variants, such as, for example, linear antibodies. They can be chimeric, humanized, or totally human. Methods are known in the art with which one of skill can readily generate additional antibodies that specifically bind to CD16 and/or that interfere with at least one of its biological functions. A functional fragment or derivative of an antibody also can be used including Fab, Fab′, Fab2, Fab′2, and single chain variable regions; so long as the antibody fragment or derivative retains the desired specificity of binding it can be used.

Antibodies for use in the compositions and methods of the invention can be produced in cell culture, in phage, or in various animals, including but not limited to cows, rabbits, goats, mice, rats, hamsters, guinea pigs, sheep, dogs, cats, monkeys, chimpanzees, apes, etc. Antibodies can be tested for specificity of binding by comparing binding to appropriate antigen to binding to irrelevant antigen or antigen mixture under a given set of conditions, as is known in the art. If the antibody binds to the appropriate antigen at least 2, 5, 7, and preferably 10 times more than to irrelevant antigen or antigen mixture then it is considered to be specific. Techniques for making such partially to fully human antibodies are known in the art and any such techniques can be used.

Affimers are known in the art and can readily be made to interfere with a given function in a manner similar to antibodies; thus, the invention further provides affimers that are anti-CD16 agents that can be used in the compositions and methods of the invention.

Affimers suitable for use in the compositions and methods of the invention can block binding of IC to CD16. Such affimers include, for example, those taught by Robinson et al. (2017) PNAS (DOI 10.1073/pnas.1707856115).

In some embodiments of the compositions and methods of the invention, a CXCR4 antagonist is used, such as, for example, those described by Portella et al. (2013) PLoS One 8: e74548 and DiMaro et al. (2016) J. Med. Chem. 59: 8369-80 (herein incorporated by reference in their entirety), including but not limited to AMD3100 and Peptide R29. Thus, in some embodiments, a CXCR4 antagonist is used in the compositions and/or methods of the invention and is selected from the group consisting of Peptide R, Peptide 1, and Peptide S described by Portella et al. (Ibid.) and having the sequences Arg-Ala-[Cys-Arg-Phe-Phe-Cys], [Cys-Trp-His-Arg-Cys], and Arg-Ala-[Cys-Arg-His-Trp-Cys], respectively, wherein square brackets indicate cyclization via disulfide bridge. In some embodiments, a CXCR4 antagonist is used in the methods of the invention and is an analog of such peptides as taught in DiMaro et al. (Ibid.)

The correlation between NK cells and stimulation by DCs in the presence of anti-CD16 antibody or other agent makes the frequency and/or change in NK cells in a patient a useful indicator of immune response. In this manner, the frequency and/or change in NK cells in a patient following a treatment can be used to assess a patient's likely clinical outcome. By monitoring the frequency and/or change in NK cells in a patient, it is possible to predict or determine whether a treatment of a patient has been or will be effective in inducing an immune response as measured, e.g., by a decrease in viral load or an extended time to viral rebound in an HIV patient, or by a clinical response in a cancer patient.

Thus, in some embodiments, by monitoring the frequency and/or change in NK cells or activated NK cells and/or monocytes in a patient treated with DCs and anti-CD16 agents or antibodies, it is also possible to evaluate when a treatment has been effective and/or when a patient has had enough doses of a treatment to be effective in inducing an immune response. In some instances, an increase of at least 20%, 30%, 40%, 50%, 60%, 100%, or 200% or more of NK cells or activated NK cells in a patient or a decrease of at least 20%, 30%, 40%, 50%, 60%, 100%, or 200% or more of monocytes will indicate that the patient has had a sufficient immune response that a treatment (e.g., treatment with AGS-004) has reached a treatment threshold and may properly be discontinued. Such treatment decisions are within the skill of a clinician with the guidance of known measures of patient health, which can include, for example, changes in patient cell populations or cytokine levels as described herein. In this manner, the invention provides methods of determining whether a treatment has been effective and/or whether a particular treatment should be continued or discontinued.

In some embodiments of the invention, the presence and/or amount of immune complexes in the blood or plasma of a patient can be evaluated as an indicator of the patient's immune response to a treatment such as, for example, treatment with DCs and/or an anti-CD16 agent or antibody. Methods of measurement are known in the art, and kits and reagents are readily commercially available, for example, as discussed in Van Hoeyveld and Bossuyt (2000) Clin. Chem. 46(2): 283-85. In some embodiments of the invention, the presence and/or amount of monocytes in the blood, plasma, or other tissue of a patient can serve as an indicator of the patient's immune response to a treatment. In some embodiments of the invention, PBMCs isolated from a patient can be cultured and evaluated for the presence and amount of monocytes in the PBMC population or for the ability of the PBMC population to kill monocytes that are added to the PBMC culture, and/or the amount of IFN-γ secreted by such cultures, for example, as described elsewhere herein and in the working examples.

Thus, in some embodiments. methods of determining or confirming effective treatment of a patient for an immune-related disease or disorder comprise the following steps in any suitable order: obtaining an aliquot of blood from the patient; quantifying the number and/or amount of one or more of NK cells, monocytes, Treg cells, ICs, and/or other indicator present in the patient's blood prior to treatment; administering a treatment to said patient comprising autologous mature DCs prepared in vitro; obtaining a post-treatment aliquot of blood from the patient; quantifying the number and/or amount of said one or more of NK cells, monocytes, Treg cells, ICs, and/or other indicator present in the patient's blood following administration of the treatment; and confirming that the number and/or amount of said one or more of NK cells, monocytes, Treg cells, and/or ICs cells present in the patient's blood has reached a treatment threshold following treatment. In some embodiments, the treatment comprising autologous mature DCs further comprises contemporaneous administration of at least one anti-CD16 antibody or other agent as described elsewhere herein.

In these methods, one of skill in the art is aware that the steps of quantifying the number and/or amount of any indicator in a patient's blood can be carried out at any time after an aliquot of blood is collected from the patient, and aliquots of blood can be stored for long periods of time (e.g., by freezing or other means of preservation) prior to quantifying an indicator. Thus, one of skill in the art appreciates that the “quantifying” steps of the methods of determining or confirming effective treatment of a patient can be carried out in any order relative to the other steps of the method so long as they occur after the aliquot of blood is collected. Thus, in some embodiments. methods of determining or confirming effective treatment of a patient for an immune-related disease or disorder comprise obtaining an aliquot of blood from the patient; administering a treatment to said patient comprising autologous mature DCs prepared in vitro: and obtaining a post-treatment aliquot of blood from the patient. wherein at some point in time after said aliquots of blood are obtained from the patient, the methods further comprise quantifying the number and/or amount of NK cells, monocytes, Treg cells, ICs, and/or other indicator present in the aliquots of blood and confirming that the number and/or amount of NK cells, monocytes, Treg cells, and/or ICs cells present in the patient's blood has reached a treatment threshold following treatment. In some embodiments, the treatment comprising autologous mature DCs further comprises contemporaneous administration of at least one anti-CD16 antibody or other agent as described elsewhere herein. In some embodiments, the methods comprise quantifying the number and/or amount of NK cells, monocytes, Treg cells, ICs, and/or other indicator present in the patient's blood aliquot obtained following treatment, but not quantifying said indicator in the patient's blood aliquot obtained prior to treatment.

A treatment threshold for NK cells, for example, can be an increase of at least 50%, 100%, 150%, or 200% of the number or frequency of NK cells present in the patient's blood, where the treatment threshold indicates that a treatment has been effective and may be discontinued. Conversely, if the number or frequency of NK cells in the patient's blood has not reached the treatment threshold, additional treatment(s) are indicated, such as, for example, additional doses of DCs and/or anti-CD16 antibody or other anti-CD16 agent. Other treatment thresholds or measures can also be used, such as, for example, RECIST values or HIV RNA assays, as appropriate.

A treatment threshold for Treg cells, for example, can be a decrease of at least 10%, 20%, 30%, 40%, 50%, 60%, 70%, 80%, 90%, 95%, or 99% or more of the number or frequency and/or activity of Treg cells present in a patient's blood or other tissue following treatment. Activity of Tregs can be assessed, for example, by measuring their ability to suppress the proliferation of effector T cells, by measuring the demethylation status of the Treg-specific demethylation region (“TSDR”), and/or by evaluating culture supernatants for IFN-γ and/or TGF-β1 following coculture with effector cells, for example as taught in Santagata et al. (2017) Oncotarget 8: 77110-20.

In some embodiments, the desired result is that the immune response has been stimulated so that an increase in functional CD4 helper T cells can be measured (that is, it is above the level of detection, such as at least 10%, 20%, or 30% or more); in these embodiments, a treatment is determined to be effective if such an increase is observed following treatment.

The IFN-γR agonist used in the PME-CD40L DC maturation process can be IFN-γ or a biologically active fragment thereof. Preferably, the IFN-γ is a mammalian IFN-γ, most preferably a human IFN-γ. The cDNA and amino acid sequence of human IFN-γ are shown in SEQ ID NOs: 5 and 6 of WO2007117682, respectively. Preferably, the IFN-γ has the sequence shown in SEQ ID NO:6 of WO2007117682, or a fragment thereof. In one embodiment, the IFN-γR comprises a polypeptide having at least 80% sequence identity with SEQ ID NO:6 of WO2007117682. Preferably, the IFN-γR agonist has at least 85%, 90%, 95%, 97%, 98% or 99% sequence identity with SEQ ID NO:6 of WO2007117682. Methods for testing the activity of IFN-γR agonists are known in the art (see, for example, Magro et al. (2004) Br. J. Pharmacol. 142: 1281-92). Immature DCs can be signaled by adding an IFN-γR agonist the culture medium, or by expressing the IFN-γR agonist in the dendritic cell. In some embodiments, the DC is transfected with an mRNA encoding an IFN-γR agonist, such as SEQ ID NO:6 of WO2007117682, or a biologically active fragment thereof. Signaling would then occur upon translation of the mRNA within the dendritic cell. In some embodiments, the IFN-γR agonist is added to the culture medium containing immature DCs and the culture medium further comprises PGE₂ and/or GM-CSF plus IL-4 or IL-13.

The second signal used to produce PME-CD40L DCs is a transient signal with a CD40 agonist. The signal can be considered transient if the DCs are loaded with an mRNA encoding a CD40 agonist, or if medium containing a CD40 agonist is removed from the DCs. Thus, persistent expression of a CD40 agonist polypeptide, such as constitutive expression of CD40L from a lentiviral vector, is not considered transient expression. The CD40 agonist signal can also be considered transient if the DCs are loaded/transfected with or with an expression vector encoding a CD40 agonist, provided that either: 1) the promoter driving CD40 agonist expression is not constitutive in DCs, or 2) the expression vector does not integrate into the DC genome or otherwise replicate in DCs.

In some methods, the CD40 agonist is a CD40L polypeptide or a CD40 agonistic antibody. In general, ligands that bind CD40 may act as a CD40 agonist, for example, a CD40 agonist can be an aptamer that binds CD40. Preferably, the CD40 agonist is delivered as mRNA encoding CD40L. Administration of the second signal comprising CD40L to the cells by transfection of immature or mature DCs with CD40L mRNA produces the modified PME-CD40L DCs that induce immunostimulatory responses rather than immunosuppressive ones.

In some methods used to produce PME-CD40L DCs, CD40L-mRNA-transfected dendritic cells are cultured in medium containing IFN-γ (and optionally PGE₂) immediately after transfection and thus prior to translation of the CD40L mRNA to produce an effective amount of a CD40L signal. In this situation, although IFN-γ is added after transfection with CD40L mRNA, the dendritic cells receive the IFN-γ signal prior to the signal that results from the translation of the CD40L mRNA. Thus, the order in which the agents are delivered to the cells is important only in that CD40L signaling must occur after IFN-γ signaling. In these methods, the signaling of the DCs can occur in vivo or ex vivo, or alternatively one or more signaling step may occur ex vivo and the remaining steps of the method can occur in vivo.

As used herein, “CD40 Ligand” (CD40L) encompasses any polypeptide or protein that specifically recognizes and activates the CD40 receptor and activates its biological activity. The term includes transmembrane and soluble forms of CD40L. In preferred embodiments, the CD40 agonist is a mammalian CD40L, preferably a human CD40L. A human CD40L cDNA and the corresponding amino acid sequence are shown in SEQ ID NOs:1 and 2 of WO2007117682, respectively.

In some methods used to prepare PME-CD40L DCs, the method comprises the sequential steps of: (a) signaling isolated immature dendritic cells (iDCs) with a first signal comprising an interferon gamma receptor (IFN-γR) agonist and a TNF-αR agonist, to produce IFN-γR-agonist-signaled dendritic cells; and (b) signaling said IFN-γR-agonist-signaled dendritic cells with a second transient signal comprising an effective amount of a CD40L polypeptide to produce CD83⁺ CCR7⁺ mature dendritic cells, wherein the CD40L polypeptide consists essentially of amino acid residues 21-261 of SEQ ID NO:2 of WO2007117682 or a polypeptide having at least 80% sequence identity to amino acid residues 21-261 of SEQ ID NO:2 of WO2007117682.

In some methods used to prepare PME-CD40L DCs, the method comprises the sequential steps of: (a) culturing isolated immature dendritic cells (iDCs) with an interferon gamma receptor (IFN-γR) agonist in the presence of a TNF-αR agonist and PGE₂ for approximately 12 to 30 hours to produce CD83⁺ mature dendritic cells; and (b) approximately 12 to 30 hours after initiating step (a), transfecting said CD83′ mature dendritic cells (mDCs) with mRNA encoding a CD40L polypeptide consisting of amino acid residues 21-261 of SEQ ID NO:2 of WO2007117682 and an mRNA encoding one or more antigens to produce CD83⁺ CCR7⁺ mature dendritic cells.

CD40 Ligand was cloned in 1993 and reported by Gauchat et al. (1993) FEBS Lett. 315: 259. Shorter soluble forms of the cell-associated full-length 39 kDa form of CD40 Ligand have been described with molecular weights of 33 kDa and 18 kDa (Graf et al. (1995) Eur. J. Immunol. 25: 1749: Ludewig et al. (1996) Eur. J. Immunol. 26: 3137; Wykes et al. (1998) Eur. J. Immunol. 28: 548). The 18 kDa soluble form generated via intracellular proteolytic cleavage lacks the cytoplasmic tail, the transmembrane region, and parts of the extracellular domain, but conserves the CD40 binding domain and retains the ability to bind to CD40 receptor; therefore, it is an example of a CD40-receptor-signaling agent. See Graf et al. (1995) supra. U.S. Pat. Nos. 5,981,724 and 5,962,406 also disclose DNA sequences encoding human CD40 Ligand (CD40L), including soluble forms of CD40L. In some methods of maturation, the CD40L polypeptide is a polypeptide comprising the sequence set forth in SEQ ID NO:2 of WO2007117682. In various embodiments, any polypeptide fragment of the full-length CD40L (or DNA or RNA encoding it) may be used in the methods or compositions of the invention provided that the polypeptide acts as a CD40 ligand by specifically binding CD40 and producing biological activity. In some methods of PME-CD40L DC maturation, CD40L is delivered to the cell as mRNA.

The method used to produce PME-CD40L DCs can also include delivering to the immature or mature DCs an effective amount of at least one antigen which will be then be processed and presented by the mature DCs. Antigen(s) can be naturally occurring or recombinantly produced. The antigens can be delivered to the cells as polypeptides or proteins or as nucleic acids (i.e., polynucleotides or genes) encoding them using methods known in the art. In methods where the antigen is delivered as a polynucleotide or gene encoding the antigen, expression of the gene results in antigen production either in the individual being treated (when delivered in vivo) or the cell culture system (when delivered in vitro); suitable techniques are known in the art. Most preferably, the polynucleotide is an mRNA.

In some methods, one or more polynucleotides encoding one or more antigens are introduced into iDCs, signaled DCs or CCR7⁺ mature DCs by methods known to those of skill in the art such as, for example, electroporation, calcium phosphate precipitation, or microinjection. Any suitable vector or delivery method may be used and can readily be selected by one of skill in the art. In preferred embodiments, the antigen or antigen-encoding mRNA is introduced together with an mRNA encoding a CD40 agonist (such as a CD40L polypeptide) or substantially concurrent with CD40 agonist signaling.

An antigen can be a single known antigen or can be a collection of known or unknown antigens; by “unknown” in this context is intended that the antigens have not been specifically individually identified. A collection of antigens may come from one particular source, such as for example a patient's cancer cells, or may come from several sources, such as, for example, HIV-infected cells from several different patients. Antigens for use in methods of producing PME-CD40L DCs include, but are not limited to, antigens from: pathogens, pathogen lysates, pathogen extracts, pathogen polypeptides, viral particles, bacteria, proteins, polypeptides, cancer cells, cancer cell lysates, cancer cell extracts, and cancer-cell-specific polypeptides. For example, antigens that can be used to produce PME-CD40L DCs include such well-known antigens as MART-1.

The amount of antigen to be employed in any particular composition or application will depend on the nature of the particular antigen and of the application for which it is being used, as will readily be appreciated by those of skill in the art, and can be adjusted by one of skill in the art to provide the necessary amount of expression. In some embodiments, the antigen is from a cancer cell or a pathogen. The cancer cell can be any type of cancer cell, including a renal cancer cell (e.g., from renal cell carcinoma), a multiple myeloma cell or a melanoma cell. Preferred pathogens include HIV and HCV. In some embodiments, the antigen is delivered to the DC in the form of RNA isolated or derived from a cancer cell or a pathogen, or from a cell infected with a pathogen. Methods for RT-PCR of RNA extracted from any cell (e.g., a cancer cell or pathogen cell), and in vitro transcription are disclosed in WO2006031870 (Nicolette et al.) and U.S. Pub. 20070248578 (Tcherepanova et al.), the contents of which are incorporated herein by reference in their entirety.

In some embodiments, an effective amount of a cytokine and/or co-stimulatory molecule is delivered to the cells or patient, in vitro or in vivo. These agents can be delivered as polypeptides, proteins or alternatively, as the polynucleotides or genes encoding them. Cytokines, co-stimulatory molecules and chemokines can be provided as impure preparations (e.g., isolates of cells expressing a cytokine gene, either endogenous or exogenous to the cell) or in a “purified” form. Purified preparations are preferably at least about 90% pure, or alternatively, at least about 95% pure, or yet further, at least about 99% pure. Alternatively, genes encoding the cytokines or inducing agents may be provided, so that gene expression results in cytokine or inducing agent production either in the individual being treated or in another expression system (e.g., an in vitro transcription/translation system or a host cell) from which expressed cytokine or inducing agent can be obtained for administration to the individual.

The immunogenicity of the cells produced by the methods of the invention can be determined by well known methodologies including, but not limited to those practiced in the working examples as well as the following:

Cytokine-release assay. Analysis of the types and quantities of cytokines secreted by cells upon contacting APCs can be a measure of functional activity. Cytokines can be measured by ELISA or ELISPOT assays to determine the rate and total amount of cytokine production, for example, as discussed in Fujihashi et al. (1993) J. Immunol. Meth. 160:181; Tanquay and Killion (1994) Lymphokine Cytokine Res. 13: 259.

Proliferation Assays. T cells will proliferate in response to reactive compositions. Proliferation can be monitored quantitatively by measuring, for example, ³H-thymidine uptake, for example, as discussed in Caruso et al. (1997) Cytometry 27: 71.

CTL induction. Mature DCs electroporated with mRNAs can be co-cultured with CD8 purified T-cells. For the first three days the cells are cultured in media supplemented with IL-2 and IL-7; on day 4, the media is supplemented with IL-2. On day 7, the CD8⁺ cells are harvested and restimulated with DC in media supplemented with IL-2 and IL-7. CTL assays are performed 3 days after the second or third stimulation.

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but do not exclude others. “Consisting essentially of” when used to define compositions and methods shall mean excluding other elements of any essential significance to the combination. Thus, a composition consisting essentially of the elements as defined herein would not exclude trace contaminants from isolation, purification, and/or culturing as well as pharmaceutically acceptable carriers, such as phosphate buffered saline, preservatives, and the like. Polypeptides or protein that “consist essentially of” a given amino acid sequence are defined herein to contain no more than three, preferably no more than two, and most preferably no more than one additional amino acids at the amino and/or carboxy terminus of the protein or polypeptide. Nucleic acids or polynucleotides that “consist essentially of” a given nucleic acid sequence are defined herein to contain no more than ten, preferably no more than six, more preferably no more than three, and most preferably no more than one additional nucleotide at the 5′ or 3′ terminus of the nucleic acid sequence. “Consisting of” shall mean excluding more than trace elements of other ingredients and substantial method steps for administering the compositions of this invention. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated, that the reagents described herein are merely exemplary and that equivalents of such are known in the art.

The term “antigen” is well understood in the art and includes any substance which is immunogenic, i.e., an immunogen. It will be appreciated that the use of any antigen is envisioned for use in the present invention and thus includes but is not limited to a self-antigen (whether normal or disease-related), an antigen of an infectious agent (e.g., a microbial antigen, viral antigen, etc.), or some other foreign antigen (e.g., a food component, pollen, etc.). The term “antigen” or “immunogen” applies to collections of more than one immunogen, so that immune responses to multiple immunogens may be modulated simultaneously. Moreover, the term includes any of a variety of different formulations of immunogen or antigen.

A “native” or “natural” or “wild-type” antigen is a polypeptide, protein or a fragment thereof which contains an epitope, which has been isolated from a natural biological source, and which can specifically bind to an antigen receptor when presented in a subject as an MHC/peptide complex, such as, for example, a T cell antigen receptor (TCR).

The term “tumor associated antigen,” “tumor antigen,” or “TAA” refers to an antigen that is associated with a tumor. Examples of well-known TAAs include gp100, MART and MAGE. Other tumor antigens may be specific to a particular tumor in a particular patient.

The terms “major histocompatibility complex” or “MHC” refers to a complex of genes encoding cell-surface molecules that are required for antigen presentation to T cells and for rapid graft rejection. In humans, the MHC is also known as the “human leukocyte antigen” or “HLA” complex. The proteins encoded by the MHC are known as “MHC molecules” and are classified into Class I and Class II MHC molecules. Class I MHC molecules are expressed by nearly all nucleated cells and have been shown to function in antigen presentation to CD8⁺ T cells. Class I molecules include HLA-A, B, and C in humans. Class II MHC molecules are known to function in CD4⁺ T cells and, in humans, include HLA-DP, -DQ, and -DR.

The term “antigen presenting cells (APCs)” refers to a class of cells capable of presenting one or more antigens in the form of peptide-MHC complex recognizable by specific effector cells of the immune system, and thereby are capable of inducing an effective cellular immune response against the antigen or antigens being presented. APCs can be intact whole cells such as macrophages, B-cells, endothelial cells, activated T-cells, and dendritic cells; or other molecules, naturally occurring or synthetic, such as purified MHC Class I molecules complexed to β2-microglobulin. While many types of cells may be capable of presenting antigens on their cell surface for T-cell recognition, only dendritic cells have the capacity to present antigens in an efficient amount to activate naive T-cells for cytotoxic T-lymphocyte (CTL) responses.

The term “dendritic cells” (herein also referred to as “DCs”) refers to a diverse population of morphologically similar cell types found in a variety of lymphoid and non-lymphoid tissues (see, e.g., Steinman (1991)Ann. Rev. Immunol. 9: 271-296). Dendritic cells constitute the most potent and preferred APCs in the organism. DCs can be differentiated from monocytes but are phenotypically distinct from monocytes; for example, CD14 antigen is not found in dendritic cells but is possessed by monocytes. Also, mature dendritic cells are not phagocytic, whereas monocytes are strongly phagocytosing cells. It has been shown that mature DCs can provide all the signals necessary for T cell activation and proliferation.

The term “immune effector cells” refers to cells capable of binding an antigen and which mediate an immune response. These cells include, but are not limited to, T cells, B cells, monocytes, macrophages, NK cells, and cytotoxic T lymphocytes (CTLs), for example CTL lines, CTL clones, and CTLs from tumor, inflammatory, or other infiltrates.

A “naïve” immune effector cell is an immune effector cell that has never been exposed to an antigen capable of activating that cell. Activation of naive immune effector cells requires both recognition of the peptide:MHC complex and the simultaneous delivery of a costimulatory signal by a professional APC in order to proliferate and differentiate into antigen-specific armed effector T cells.

As used herein, the term “educated, antigen-specific immune effector cell” is an immune effector cell as defined above which has previously encountered an antigen. In contrast to its naïve counterpart, activation of an educated, antigen-specific immune effector cell does not require a costimulatory signal; recognition of the peptide:MHC complex is sufficient.

“Immune response” broadly refers to the antigen-specific responses of lymphocytes to foreign substances. Any substance that can elicit an immune response is said to be “immunogenic” and is referred to as an “immunogen.” An immune response of this invention can be humoral (via antibody activity) or cell-mediated (via T cell activation), and can be detected or monitored by any of a number of methods known in the art, including for example the production of certain antibodies, cytokines, or cell types as described elsewhere herein.

“Immune Complex” or “Immunoglobulin Complex” (also “IC”) refers to an antibody bound to an antigen. In some embodiments, the IC comprises an IgG antibody. In the IC, the bound antigen and antibody act as a unitary object and the molecule formed has its own specific epitope(s). In a subject, Immune Complexes can be found in circulation (i.e., in the circulating blood), and can also be formed in vitro, as is known in the art.

“Activated,” when used in reference to a T cell or NK cell implies that the cell is no longer in Go phase, and begins to produce one or more of cytotoxins, cytokines and other related membrane-associated proteins characteristic of the cell type, and is capable of recognizing and binding any target cell that displays the particular peptide/MHC complex on its surface, and releasing its effector molecules, as described, for example, in Fogel et al. (2013) J. Immunol. 190: 6269-76 and Shipkova and Wieland (2012) Clin. Chim. Acta 413: 1338-49.

As used herein, the term “inducing an immune response in a subject” is a term understood in the art and refers to an increase of at least about 2-fold, or at least about 5-fold, or at least about 10-fold, or at least about 100-fold, or at least about 500-fold, or at least about 1000-fold or more in an immune response to an antigen which can be detected or measured, after introducing the antigen into the subject, relative to the immune response (if any) before introduction of the antigen into the subject. An immune response to an antigen can include but is not limited to: production of an antigen-specific antibody; production of an immune cell expressing on its surface a molecule which specifically binds to an antigen; and an increase in cytokine production. Methods of determining whether an immune response to a given antigen has been induced are well known in the art. For example, antigen-specific antibody can be detected using any of a variety of immunoassays known in the art, including, but not limited to, ELISA, wherein, for example, binding of an antibody in a sample to an immobilized antigen is detected with an appropriately labeled second antibody (e.g., enzyme-labeled mouse anti-human Ig antibody).

By “treatment indicator” or “immune indicator” or “indicator” is intended a measurable blood chemistry component or cell type that is known to respond to stimulation or modulation of the immune system in a subject, either in response to genetic or environmental stimuli or in response to treatment, such as, for example, the compositions and methods of the present invention. In some embodiments, a “treatment indicator” or “immune indicator” or “indicator” is selected from: a change in the frequency of NK cells in the subject's peripheral blood; an increase in activated NK cells following treatment that is correlated with an increase in PD-1⁺ CD4 T cells; and PBMCs extracted from a subject following treatment that exhibit a decrease in their ability to kill monocytes in an in vitro coculture in comparison to PBMCs extracted from said subject prior to treatment, or earlier in a course of treatment.

As used herein, the term “cytokine” refers to any one of the numerous factors that exert a variety of effects on cells, for example, inducing growth or proliferation. Non-limiting examples of cytokines which may be used alone or in combination in the practice of the present invention include interleukin-2 (IL-2), stem cell factor (SCF), interleukin-3 (IL-3), interleukin-6 (IL-6), interleukin-12 (IL-12), G-CSF, granulocyte macrophage-colony stimulating factor (GM-CSF), interleukin-1 alpha (IL-1α), interleukin-1L (IL-1L), MIP-11, leukemia inhibitory factor (LIF), c-kit ligand, thrombopoietin (TPO), IL-15, and 1L-17. Cytokines are readily commercially available and may be ‘natural’ purified cytokines or may be recombinantly produced.

The terms “polynucleotide,” “nucleic acid,” and “nucleic acid molecule” are used interchangeably to refer to polymeric forms of nucleotides of any length. The polynucleotides may contain deoxyribonucleotides, ribonucleotides, and/or their analogs. The term “polynucleotide” includes, for example, a gene or gene fragment, plasmids, vectors, isolated DNA of any sequence, isolated RNA of any sequence, nucleic acid probes, and primers. In addition to a native nucleic acid molecule, a nucleic acid molecule of the present invention may also comprise modified nucleic acid molecules. As used herein, mRNA refers to an RNA that can be translated in a dendritic cell. Such mRNAs typically are capped and have a ribosome binding site (Kozak sequence) and a translational initiation codon. As used herein, an RNA corresponding to a cDNA sequence refers to an RNA sequence having the same sequence as the cDNA sequence, except that the nucleotides are ribonucleotides instead of deoxyribonucleotides, as thymine (T) base in DNA is replaced by uracil (U) base in RNA.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D and L optical isomers, amino acid analogs and peptidomimetics. A peptide of three or more amino acids is commonly called an oligopeptide if the peptide chain is relatively short, whereas if the peptide chain is long, the peptide is commonly called a polypeptide or a protein.

A “conservative alteration” to a polypeptide or protein is one that results in an alternative amino acid of similar charge density, hydrophilicity or hydrophobicity, size, and/or configuration (e.g., Val for Ile). In comparison, a “nonconservative alteration” is one that results in an alternative amino acid of differing charge density, hydrophilicity or hydrophobicity, size and/or configuration (e.g., Val for Phe). The means of making such modifications are well-known in the art.

The term “genetically modified” means containing and/or expressing a foreign gene or nucleic acid sequence which in turn modifies the genotype or phenotype of the cell or its progeny. In other words, it refers to any addition, deletion or disruption to a cell's endogenous nucleotides.

As used herein, “expression” refers to the processes by which polynucleotides are transcribed into mRNA and mRNA is translated into peptides, polypeptides, or proteins. If the polynucleotide is derived from genomic DNA of an appropriate eukaryotic host, expression may include splicing of the mRNA. Regulatory elements required for expression are known in the art and include promoter sequences to bind RNA polymerase and transcription initiation sequences for ribosome binding. Appropriate vectors for bacterial and/or eukaryotic expression are known in the art and are available commercially.

“Under transcriptional control” is a term understood in the art and indicates that transcription of a polynucleotide sequence (usually a DNA sequence) depends on its being operatively linked to an element which contributes to the initiation of, or promotes. transcription. “Operatively linked” refers to a juxtaposition wherein the elements are in an arrangement allowing them to function.

“Gene delivery,” “gene transfer,” “transfection,” and the like as used herein are terms referring to the introduction of an exogenous polynucleotide into a host cell, regardless of the method used for the introduction. The introduced polynucleotide can be stably maintained in the host cell or may be transiently expressed. In preferred embodiments, an mRNA is introduced into a target cell and is transiently expressed. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are capable of mediating transfer of genes to mammalian cells and are known in the art.

The sequence of a polynucleotide or portion thereof (or a polypeptide or portion thereof) has a certain percentage of “sequence identity” to another sequence (for example, 80%, 85%, 90%, or 95%) when that percentage of bases or amino acids are the same when the two sequences are aligned and compared. The proper alignment and the percent sequence identity between two sequences can be determined using one of the well-known and publicly available BLAST alignment programs with default parameters.

With regard to molecules, the term “isolated” means separated from constituents, cellular and otherwise, with which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. A mammalian cell such as dendritic cell is isolated from an organism if it is removed from the anatomical site from which it is found in an organism. In addition, a “concentrated,” “separated,” or “diluted” polynucleotide, peptide, polypeptide, protein, antibody, or fragment(s) thereof is distinguishable from its naturally occurring counterpart in that the concentration or number of molecules per volume is greater when “concentrated” or less when “separated” or “diluted” than the concentration or number of molecules per volume of its naturally occurring counterpart.

“Host cell,” “target cell,” or “recipient cell” are intended to include any individual cell or cell culture which can be or has been recipient(s) for vectors or the incorporation of exogenous nucleic acid molecules, polynucleotides and/or proteins. These terms are also intended to include progeny of a single cell. The cells may be prokaryotic or eukaryotic, and include but are not limited to bacterial cells, yeast cells, animal cells, and mammalian cells. The term “culturing” refers to the in vitro maintenance, differentiation, and/or propagation of cells in suitable media.

A “subject” or “patient” is a mammal; in some embodiments, a patient is a human patient. A subject or patient can also be any other mammal, including a monkey or ape, or any domestic animal such as a dog, cat, horse, etc.

By “cancer” is meant the abnormal presence of cells which exhibit relatively autonomous growth, so that a cancer cell exhibits an aberrant growth phenotype characterized by a significant loss of cell proliferation control (i.e., it is neoplastic). Cancerous cells can be benign or malignant. In various embodiments, cancer affects cells of the bladder, blood, brain, breast, colon, digestive tract, lung, ovaries, pancreas, prostate gland, or skin. The definition of a cancer cell, as used herein, includes not only a primary cancer cell, but also any cell derived from a cancer cell ancestor, including metastasized cancer cells, in vitro cultures, and cell lines derived from cancer cells. Cancer includes, but is not limited to, solid tumors, liquid tumors, hematologic malignancies, renal cell cancer, melanoma, breast cancer, prostate cancer, testicular cancer, bladder cancer, ovarian cancer, cervical cancer, stomach cancer, esophageal cancer, pancreatic cancer, lung cancer, neuroblastoma, glioblastoma, retinoblastoma, leukemias, myelomas, lymphomas, hepatoma, adenomas, sarcomas, carcinomas, blastomas, etc. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition. In some embodiments, RECIST criteria are used to evaluate cancer in a patient.

By “enriched” is meant a composition comprising a particular cell type in a greater percentage of total cells than is found in another composition, such as, for example, the tissue(s) where the particular cell type is present in an organism or a group or mixture of cells in which the particular cell type was previously present. For example, in an “enriched” culture or preparation of NK cells made by the methods of the invention, NK cells are present in a higher percentage of total cells as compared to an in vitro culture in which they were produced or cultured, or as compared to a patient tissue in vivo, such as, for example, blood or tumor tissue. In some embodiments, NK cells that are in an “enriched” composition are present as more than 10%, 20%, 30%, 40%, 50%, 60%, or 70% of the cells in that composition. By “purified” or “isolated” is intended that a particular cell type (e.g., NK cells) is present as more than 30%, 40%, 50%, 60%, 70%, 800%, 90%, or 95% or 99% of the cells in that composition. By “depleted” is intended that the frequency of that cell type is decreased in a particular composition or population of cells, e.g., by at least 10%, 20%, 30%, 40%, 50%, 60%, 70%, 80%, or 90% or more, or 100%. In using the terms “enriched,” “purified,” or “isolated,” the particular cell type can be defined by any one or more cell surface markers, as appropriate under the circumstances.

The term “pharmaceutical composition” or “medicament” is intended to include the combination of an active agent with a carrier, inert or active, making the composition suitable for diagnostic or therapeutic use in vitro or in vivo. The term “pharmaceutically acceptable carrier” encompasses any suitable pharmaceutical carrier, such as, for example, a phosphate buffered saline solution, water, and emulsions, such as an oil/water or water/oil emulsion, and various types of wetting agents. A “pharmaceutical composition” or “medicament” also can include stabilizers and preservatives. For examples of carriers, stabilizers and adjuvants, see Martin, Remington's Pharmaceutical Sciences, 18th Ed. (Mack Publ. Co., Easton (1990)).

An “effective amount” of a composition is an amount sufficient to produce beneficial or desired results, such as enhanced immune response, treatment, prevention or amelioration of a medical condition (disease, infection, etc), or increase or decrease in a treatment indicator, as appropriate. An effective amount can be administered in one or more administrations, applications, or dosages. Suitable dosages will vary depending on body weight, age, health, disease or condition to be treated and route of administration; methods of determining an effective amount are known in the art. In some embodiments, a synergistic effect of more than one treatment or type of treatment can be detected or identified as a decrease in the amount of a particular (first) composition required to constitute an “effective amount” when used contemporaneously or simultaneously with another (second) composition as compared to using the (first) composition alone (i.e., without the second composition).

As used herein, “signaling” means contacting an immature or mature dendritic cell with an IFN-γ receptor agonist, a TNF-α receptor agonist, and/or a CD40L polypeptide or other CD40 agonist. Such agonists can be provided externally (e.g., in the cell culture medium) or, for example, a polypeptide agonist can be provided via transfection of an immature or mature dendritic cell with a nucleic acid encoding the polypeptide. In cases where the polypeptide(s) is provided by transfecting a dendritic cell with a nucleic acid encoding the polypeptide, signaling is effected upon translation of an mRNA encoding the polypeptide, rather than upon transfection with the nucleic acid. As used herein, the term “mature dendritic cells” means dendritic cells that demonstrate elevated cell surface expression of co-stimulator molecule CD83 compared to immature DCs (iDCs), or having particular cell surface markers as described elsewhere herein.

Throughout this disclosure, various publications, patents and published patent specifications are referenced by an identifying citation. The disclosures of these publications, patents and published patent specifications are hereby specifically incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are known in the art and explained in the literature. In accordance with the above description, the following examples are intended to illustrate, but not limit, the various aspects of this invention.

### EXPERIMENTAL EXAMPLES

### Example 1—PME-CD40L DC Maturation Process and Evaluation

PME-CD40L DCs were prepared essentially as described in Calderhead et al. ((2008) J. Immunother. 31: 731-41). Briefly, CD40L was cloned from activated T cells that had been stimulated with phorbol 12-myristate 13-acetate (PMA); RT-PCR was performed on total RNA from the T cells using gene-specific CD40L primers to amplify and clone CD40L. Human PBMCs were isolated from leukapheresis collections from healthy volunteers by Ficoll-histopaque density centrifugation. PBMCs were resuspended in culture medium and allowed to adhere to plastic flasks, then nonadherent cells were removed and remaining cells were cultured in medium supplemented with GM-CSF (1000 U/ml) and IL-4 (1000 U/ml) for 5-6 days at 37° C., 5% CO₂. DCs were harvested, washed in PBS, re-suspended in chilled Viaspan® (DuPont Pharma®), and placed on ice. DCs were mixed with CD40L mRNA and antigen-encoding mRNA and electroporated. Immediately after electroporation, DCs were washed and re-suspended in medium that was supplemented with GM-CSF and IL-4. DCs were cultured for either 4 or 24 hours at 37° C. in low-adherence plates with additional maturation stimuli as described below.

Immature DCs were phenotypically matured on day 5 of culture by adding TNF-α, IFN-γ, and PGE₂. On day 6, DCs were harvested and electroporated with antigen and CD40L mRNA as described above, and cultured in media containing GM-CSF and IL-4 for 4 hours prior to harvest or formulation for vaccine production.

For flow cytometric analysis, DCs were harvested and re-suspended in chilled PBS/1% FCS, then mixed with phycoerythrin (PE) or FITC-conjugated antibodies specific for CD1a, CD209, human leukocyte antigen (HLA)-ABC, HLA-DR, CD80, CD86, CD38, CD40, CD25, CD123, CD83, CCR6, CCR7, CD70, and CD14; isotype-matched antibodies were used as controls. After thorough washing, fluorescence analysis was performed with a FACScalibur flow cytometer (BD Biosciences™) and CellQuest software (BD Biosciences™). Chemotaxis of DCs was measured by migration through a 8-μm pore size polycarbonate filter. IL-10 and IL-12 in the DC supernatants were determined using ELISA.

### Example 2—NK Cells as Markers of Immune Response in RCC Patients Treated with AGS-003

As part of Argos Therapeutics' phase III clinical trial (“ADAPT” trial), human RCC patients were treated with PME-CD40L DCs encoding antigens prepared essentially as described in WO2006042177 (Healey et al.); WO2007117682 (Tcherepanova el al.); DeBenedette et al. (2008) J. Immunol. 181: 5296-5305; Calderhead et al. (2008) J. Immunother. 31: 731-4; and WO 2006031870 (Nicolette et al.). In this clinical trial, patients were treated with multiple doses of autologous PME-CD40L DCs encoding RCC antigens from the subject (also referred to herein as “AGS-003” or “Rocapudencel-T” or “Roca”). Patients' immune response was monitored pre- and post-treatment.

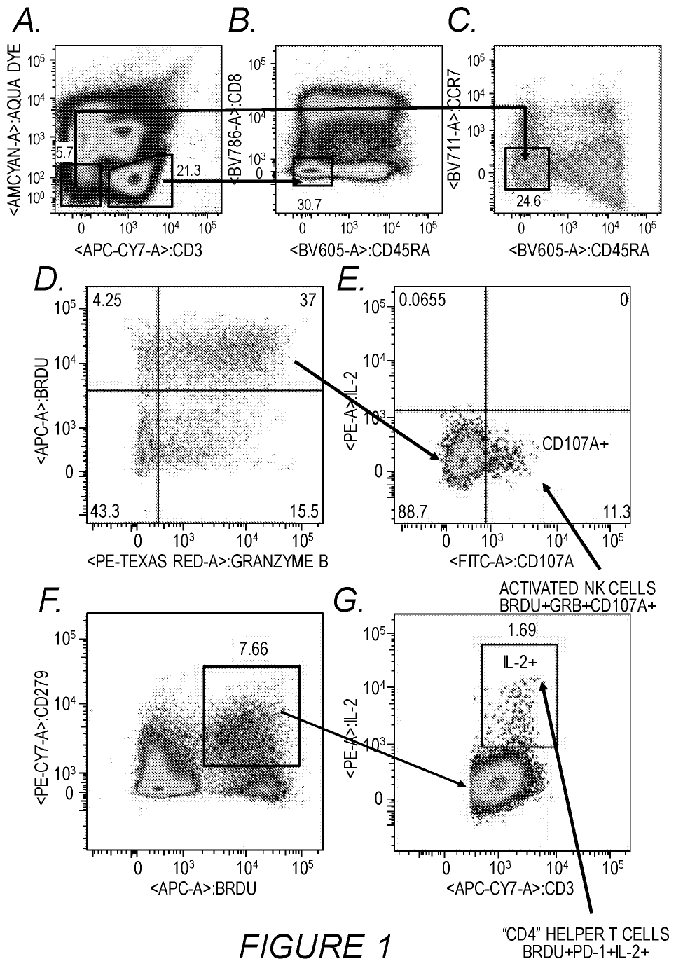

PBMCs were collected pre- and post-Roca administration from patients enrolled in the ADAPT trial. FIGS. 1A, 1B, 1C, 1D, 1E, 1F, and 1G show the identification of activated NK cells and functional CD4 T cells in mRCC patient PBMCs stimulated with autologous DC product. NK cells were identified in several steps: first, as negative for the marker CD3 (FIG. 1A); then cells within the CD3 negative gate were screened for CCR7 and CD45RA expression, with NK cells being negative for both CCR7 and CD45RA (see FIG. 1C). NK cells were identified as activated when they were BrdU⁺ and granzyme B positive (see FIG. 1D, upper right quadrant). Further subgating identified cells positive for CD107A (FIG. 1E). In this manner, activated NK cells were identified as CD3⁻/CCR7⁻/CD45RA⁻/BrdU⁺/Grb⁺/CD107a⁺.

FIG. 1 also shows the identification of functional helper CD4 T cells in mRCC patient PBMCs stimulated with autologous product. CD4 T cells were first identified as CD3 positive, CD8 negative, and CD45RA negative (FIG. 1A, FIG. 1B (boxed area and black arrow)). Within the CD8 and CD45RA negative gate (FIG. 1B), further subgating identified activated CD4 T cells, defined as PD-1⁺ BrdU⁺ (see FIG. 1F, boxed area). Functional CD4 helper T cells within the PD-1⁺/BrdU⁺ population were identified by the presence of IL-2 (FIG. 1G, boxed area). In this manner, functional helper CD4 cells were identified as CD3⁺/CD8⁻/CD45RA⁻/PD-1⁻/BrdU⁺/IL-2⁺.

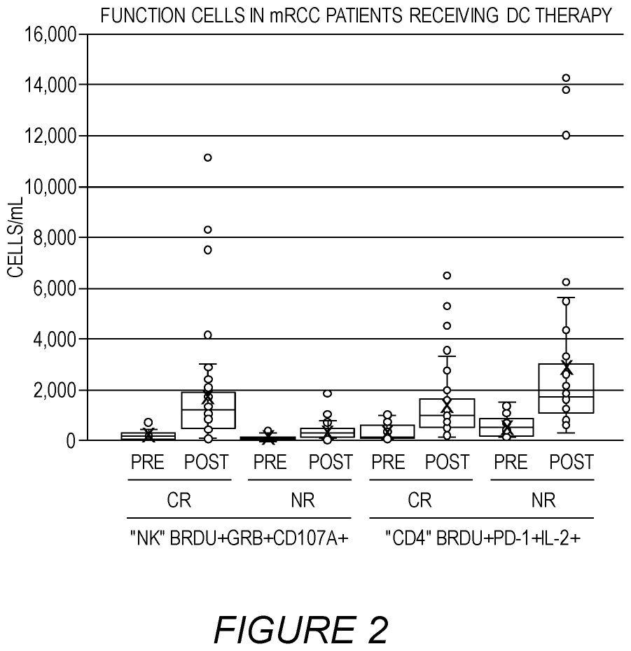

FIG. 2 shows data demonstrating that mRCC Clinical Responder (“CR”) patients to whom AGS-003 was administered exhibited higher numbers of both CD4 helper T cells and activated NK cells than Non-Responder (“NR”) patients. Cells from CR patients were compared to cells from NR patients. Five patients scored as CR according to RECIST criteria and were still alive as of an interim data analysis in February 2017. Five other patients were classified as NR with a median overall survival of less than 17.6 months and did not exhibit any clinical responses as determined using RECIST criteria. All five NR patients were deceased prior to the interim data analysis in February 2017. Patient PBMCs had been collected at two time points prior to AGS-003 administration (visits 1 and 2) and at three time points post-AGS-003 administration (visits 6, 9, and 12). Data from visits 1 and 2 were pooled as the pre-treatment data points (“Pre”) and data from visits 6, 9 and 12 were pooled as the post-treatment data points (“Post”). The number of activated NK cells and functional CD4 T cells in the patient samples were quantified using Trucount tubes during collection and analyzed using multi-color flow cytometry.

The two patient populations (CR and NR) exhibited clear differences in the number of activated NK cells following treatment with AGS-003. The CR patients showed greater numbers of activated NK cells than NR patients. Both patient groups had increases in the numbers of functional CD4 T cells.

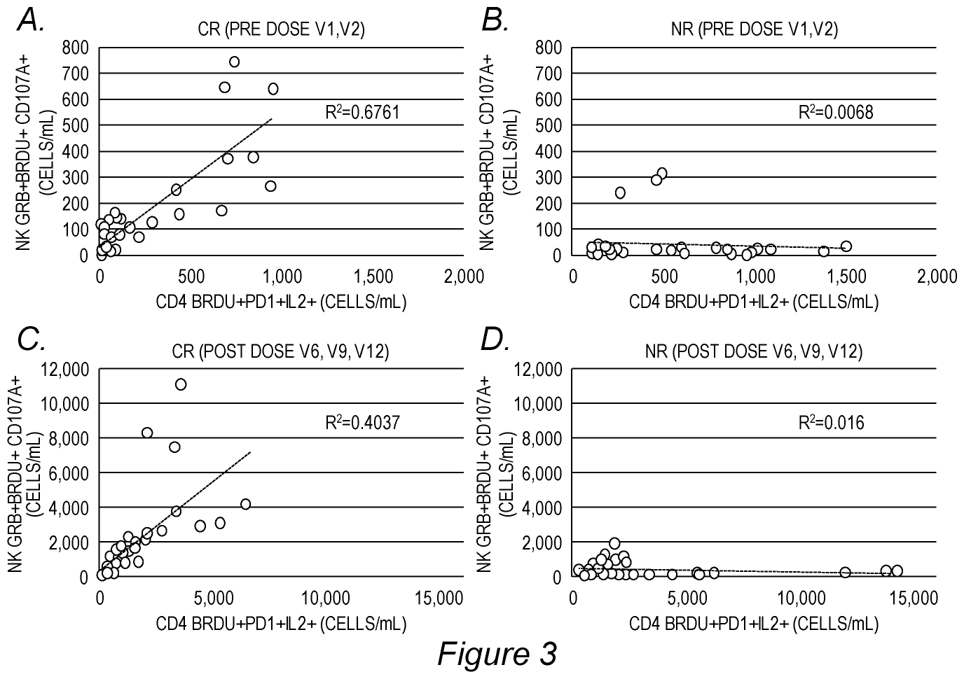

FIGS. 3A, 3B, 3C, and 3D present data showing a positive correlation between the number of activated NK cells and functional CD4 helper T cells in mRCC subjects treated with AGS-003 who had a measurable clinical response. This positive correlation was seen both pre-treatment (FIG. 3A) and post-treatment (FIG. 3C), even though the post-treatment samples showed a dramatic increase in the numbers of both cell types. In contrast, in the Non-Responder patients, no correlation was observed between activated NK cells and functional CD4 T cells either pre-treatment (FIG. 3B) or post-treatment (FIG. 3D).

### Example 3-Blocking Immune Complex Binding to CD4+ T Cells During DC Stimulation Induces Activated NK Cells

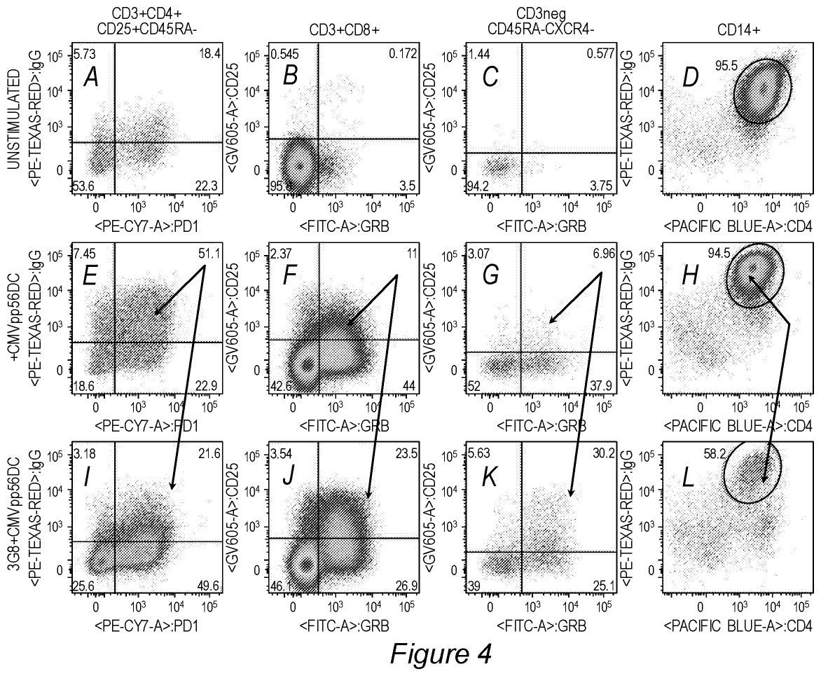

FIGS. 4A, 4B, 4C, 4D, 4E, 4F, 4G, 4H, 4I, 4, 4K, and 4L present data showing that blocking Immunoglobulin Complex (IC) binding to CD4 T cells with anti-CD16 antibody during in vitro DC stimulation induces activated NK cells. PBMC cultures were established from a normal CMV⁺ donor. Control cultures were unstimulated (FIGS. 4A, 4B, 4C, and 4D). Other cultures were stimulated with DCs electroporated with RNA encoding the pp65 protein antigen from CMV (CMVpp56DC, FIGS. 4E, 4F, 4G, and 4H), or with the combination of CMVpp56DC cells and anti-CD16 antibody (3G8, FIGS. 41, 4J, 4K, and 4L). PBMCs stimulated with CMVpp65DC showed an increased binding of immunoglobulin complexes (ICs) to the surface of activated CD4⁺ T cells (compare FIG. 4A (18.4% in upper right section) versus FIG. 4E (51.1% in upper right quadrant)).

The binding of ICs by PD-1⁺ CD4 T cells was detected by staining cells with an anti-Ig-specific antibody. In the presence of the anti-CD16 antibody 3G8, there is a blockade of IC binding to the activated CD4 T cells (compare FIG. 4E to FIG. 4I, 51.1% versus 21.6%). In addition, these cultures show an increase in activated CD8 T cells (compare FIG. 4F to 4J, CD3⁺ CD8⁺ cells double positive for CD25 and Grb increasing from 11% to 23.5%) and also in activated NK cells (compare FIG. 4G to 4K, CD3 CD45RA CXCR4 cells double positive for CD25 and Grb increasing from 6.96% to 30.2%. Moreover, cultures stimulated with the anti-CD16 antibody in addition to CMVpp65DC showed a decrease in the presence of CD14 monocytes (compare FIG. 4H to 4L, decreasing from 94.5% to 58.2%). This data suggested increased killing of monocytes in the combination cultures.

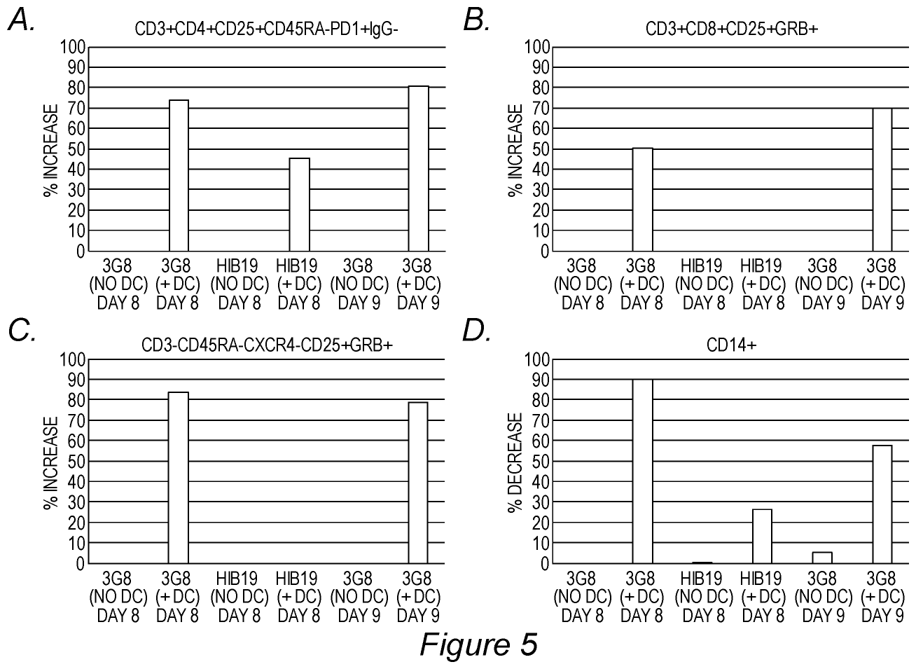

FIGS. 5A, 5B, 5C, and 5D present data showing that monocyte killing in activated cultures is specific to CD16 activation and is cell driven. Day 8 cultures were stimulated with DCs and either the anti-CD16 antibody (3G8) or a control antibody (HIB19). FIG. 5A shows the percentage increase in the cultures of activated CD4 T cells that do not bind ICs;

FIG. 5B shows the percentage increase in activated CD8 T cells; FIG. 5C shows the percent increase in activated NK cells; and FIG. 5D shows the percent decrease in CD14+ monocytes.

Cells stimulated with the anti-CD16 antibody 3G8 alone (“3G8 (noDC)”) or the control antibody HiB19 alone (“HIB19 (no DC)”) did not show an increase in activated CD4 T cells, activated CD8 T cells, or activated NK cells, and also did not show a decrease in CD14+ monocytes. In further experiments, the Day 8 cultures were washed and additional PBMCs were added containing CD14+ monocytes; these cultures were then incubated for an additional day (“Day 9”). In these “Day 9” experiments, cultures stimulated with both 3G8 and DCs maintained an increase in activated CD4 T cells (FIG. 5A), CD8 T cells (FIG. 5B) and NK cells (FIG. 5C). As shown in FIG. 5D, when fresh monocytes are added to stimulated cultures, the activated cells maintain the ability to kill the added monocytes without any additional stimulation or addition of anti-CD16 antibody.

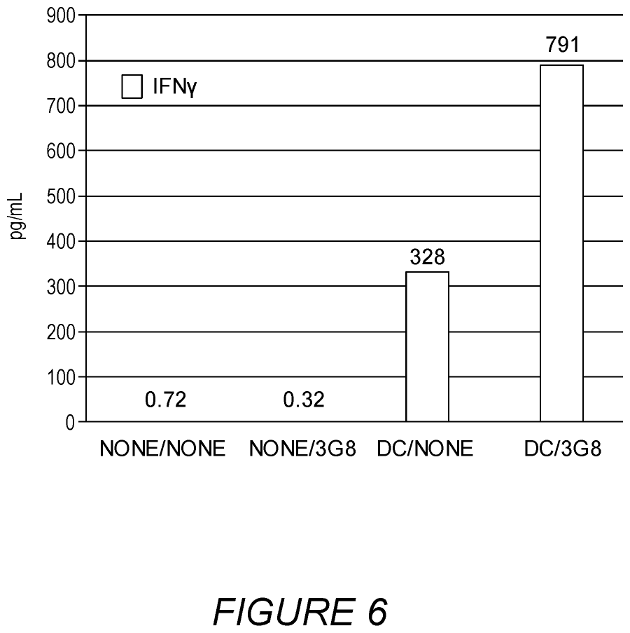

FIG. 6 presents data showing that a combination of anti-CD16 antibody and DCs increases IFN-gamma (“IFN-g”) secretion in PBMC cultures. The cultures were unstimulated (“None/none”), stimulated with 3G8 only (“None/3G8”), DCs only (“DC/none”) or a combination of 3G8 and DCs (“DC/3G8”) by cytokine bead array. The culture supernatants were collected and IFN-gamma was measured. IFN-gamma was increased in cultures stimulated with DCs and more than twice as much in cultures stimulated with the combination of DCs and anti-CD16 antibody.

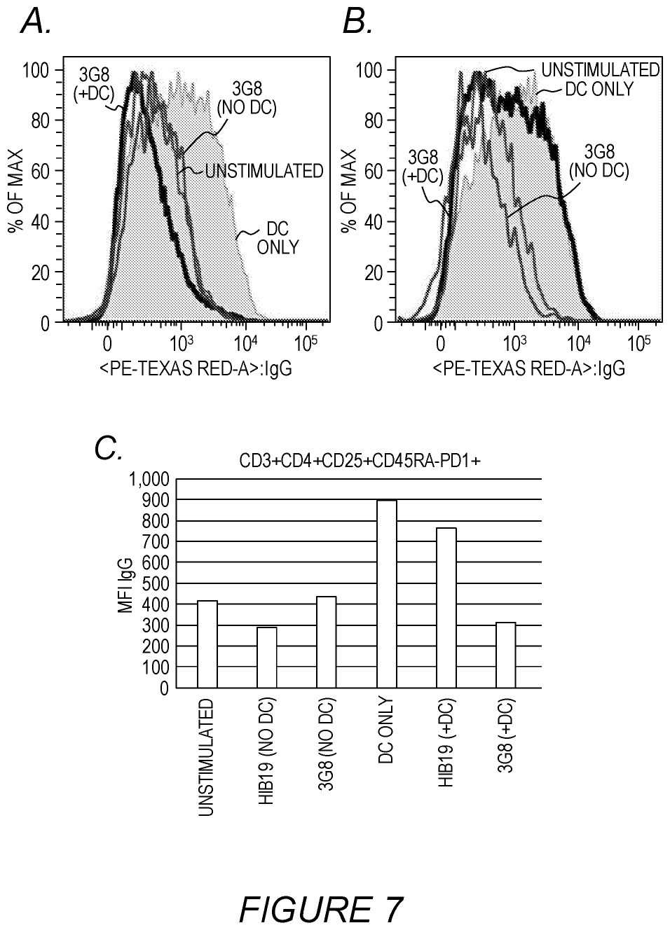

FIGS. 7A, 7B, and 7C present data showing that dendritic cell (DC) stimulation of PBMC cultures resulted in the binding of immune complexes (ICs) to the cell surface of CD4 T cells. Surface-bound ICs were detected with anti-IgG antibody (gray shading in FIGS. 7A and 7B). Unstimulated PBMCs (red lines in FIGS. 7A and 7B) or PBMCs stimulated only with antibody did not induce binding of ICs to the cell surface of CD4 T cells (either anti-CD16 antibody 3G8 in FIG. 7A or control antibody HIB19 in FIG. 7B, blue lines). However, when anti-CD16 antibody 3G8 was added to DC-stimulated cultures, ICs were blocked from binding to CD4 T cells (black line, FIG. 7A), whereas a control antibody not specific for CD16 (HIB19) did not block IC binding to the CD4 T cells (black lines in FIG. 7B), showing that the blocking of IC binding (FIG. 7A) was specific to the anti-CD16 antibody and thus that immune complexes bound specifically to CD16 on CD4 T cells.

Measuring this binding of ICs to CD4 T cells by mean fluorescence intensity (MFI, FIG. 7C) shows that anti-CD16 antibody blocks IC binding so that MFI is reduced to the background values detected on unstimulated PBMCs, down from higher values obtained when PBMCs are stimulated with DCs (FIG. 7C, “DC only”). Actual MFI values: anti-CD16 antibody blocking ICs binding, MFI value of 315; background values detected on unstimulated PBMCs, MFI value of 418; PBMCs stimulated with DCs (“DC only”), MFI value of 899.

What is claimed:

1. A method for obtaining a population of activated NK cells suitable for introduction into a human patient comprising the steps of:
a) isolating PBMCs from a human patient; and
b) culturing said PBMCs in vitro with an anti-CD16 antibody or agent and PME-CD40L mature DCs in a coculture for a time sufficient to induce an increase in the number of NK cells in said coculture.

2. The method of claim 1, wherein said PME-CD40L mature DCs are loaded with an antigen.

3. The method of claim 2, wherein said antigen is prepared from cells of an HIV-infected patient.

4. The method of claim 2, wherein said antigen is prepared from cancer cells.

5. The method of claim 1, wherein said NK cells are identified as being CD3−/CCR7−/CD45RA−/BrdU+/Grb+/CD107a+.

6. The method of claim 1, wherein said NK cells are identified as being CD3−/CD16+/CD56+.

7. The method of claim 1, wherein said NK cells are identified as being CD3−/CXCR4−/CD45RA−/Ki67+/Grb+.

8. The method of claim 1, wherein said anti-CD16 antibody or agent binds the same epitope as the 3G8 antibody.

9. A method for increasing the population of NK cells in a patient comprising the steps of:
a) administering AGS-003 to a patient; and
b) administering an anti-CD16 antibody or agent to said patient,
wherein the population of NK cells in said patient is increased.

10. A method of determining whether a treatment of a patient has been effective comprising the steps of:
a) obtaining a first aliquot of blood from a patient;
b) administering a treatment to said patient comprising autologous mature DCs prepared in vitro and an anti-CD16 antibody or agent, wherein said administering is either concurrent or sequential; and
c) after an interval of time, obtaining a second aliquot of blood from a patient, wherein a treatment is determined to have been effective if said second aliquot of blood contains one or more treatment indicator(s) selected from:
(i) an increase in the frequency of NK cells in comparison to said first aliquot of blood;
(ii) an increase in activated NK cells that is correlated with an increase in PD-1+CD4 T cells in comparison to said first aliquot of blood; and
(iii) PBMCs that exhibit a decrease in their ability to kill monocytes in an in vitro coculture in comparison to PBMCs from said first aliquot of blood.

11. The method of claim 10, wherein the increase in the frequency of NK cells is at least 50%.
